(19) United States
(12) Patent Application Publication
(10) Pub. No.: US 2023/0414869 A1
(43) Pub. Date: Dec. 28, 2023

(54) Catheter Placement System

(71) Applicant: Becton, Dickinson and Company, Franklin Lakes, NJ (US)

(72) Inventors: Jack Balji, Mahwah, NJ (US); Ashley Rachel Rothenberg, Morris Plains, NJ (US)

(21) Appl. No.: 18/036,017

(22) PCT Filed: Nov. 9, 2021
(86) PCT No.: PCT/US21/58545

§ 371 Date: May 9, 2023

Related U.S. Application Data
(60) Provisional application No. 63/113,055, filed on Nov. 12, 2020.

(51) Int. Cl. A61M 5/168; A61M 5/50

(52) U.S. Cl. CPC A61M 5/16859; A61M 5/5086; A61M 2025/0001

(57) ABSTRACT

A system may include an intravenous (IV) catheter comprising a first portion configured to be inserted into a vein of a patient and a second portion configured to remain outside a patient when the first portion is inserted into the patient. The IV catheter may include a pressure sensor configured to detect a pressure of a fluid associated with the IV catheter. A method may include receiving data associated with the pressure of the fluid associated with the IV catheter, and determining whether the IV catheter is inserted into the vein of the patient based on the pressure of the fluid associated with the IV catheter.

### CROSS-REFERENCE TO RELATED APPLICATION

The present application claims priority to U.S. Provisional Application No. 63/113,055, entitled “Catheter Placement System”, filed Nov. 12, 2020, the entire disclosure of which is hereby incorporated by reference in its entirety.

### BACKGROUND

### 1. Field

The present disclosure relates generally to catheter placement and, in some non-limiting embodiments or aspects, to a system for determining whether a catheter is inserted into a vein of a patient.

### 2. Technical Considerations

A catheter may be introduced to a patient to deliver fluid to the patient. For example, a portion of a catheter may be inserted into a vein of the patient. The catheter may then deliver fluid (saline, antibiotics, and/or the like) while the patient is conscious or not conscious. However, in some instances a tip of the catheter may be dislodged from the vein of the patient (e.g., slip out of the vein or pass through a wall of the vein), and cause fluid to be delivered to tissue outside the vein, referred to as intravenous (IV) infiltration. While some patients may be able to notify a clinician about pain associated with IV infiltration, other patients (e.g., unconscious patients, younger patients, and/or the like) may not be able to notify a clinician about pain associated with IV infiltration. As such, it is advantageous to provide systems that determine whether a catheter is inserted into a vein of a patient.

### SUMMARY

Non-limiting embodiments or aspects are set forth in the following numbered clauses:

Clause 1: A system, comprising: an intravenous (IV) catheter comprising a first portion configured to be inserted into a vein of a patient and a second portion configured to remain outside a patient when the first portion is inserted into the patient; wherein the second portion of the IV catheter comprises a pressure sensor configured to detect a pressure of a fluid associated with the second portion of the IV catheter.

Clause 2: The system of clause 1, wherein the pressure sensor is disposed within a lumen of the second portion of the IV catheter.

Clause 3: The system of clauses 1 or 2, wherein the pressure sensor is embedded in a sidewall of the second portion of the IV catheter.

Clause 4: The system of any of clauses 1-3, wherein the pressure sensor is disposed along an exterior surface of the second portion of the IV catheter.

Clause 5: The system of any of clauses 1-4, wherein, when the first portion of the IV catheter is inserted into the vein of the patient and the second portion of the IV catheter is configured to be in fluid communication with the first portion of the IV catheter and the vein of the patient.

Clause 6: The system of any of clauses 1-5, wherein the pressure sensor is configured to detect the pressure of the fluid associated with the vein of the patient based on the pressure sensor being positioned along a proximal portion of a fluid path of the patient and a signal generator being placed along a distal portion of the fluid path of the patient.

Clause 7: The system of any of clauses 1-6, wherein the pressure sensor is configured to detect the pressure of the fluid associated with the vein of the patient based on the pressure sensor being positioned along a distal portion of a fluid path of the patient and a signal generator being placed along a proximal portion of the fluid path of the patient.

Clause 8: The system of any of clauses 1-7, wherein the fluid associated with the second portion of the IV catheter comprises fluid disposed within the second portion of the IV catheter, and wherein the pressure sensor is configured to detect one or more fluctuations of the pressure of the fluid disposed within the second portion of the IV catheter based on generation of one or more signals transmitted to a second portion of the patient different from a first portion of the patient where the first portion of the IV catheter is inserted.

Clause 9: The system of any of clauses 1-8, further comprising: at least one processor programmed or configured to: receive data associated with the pressure of the fluid associated with the second portion of the IV catheter, and determine whether the IV catheter is inserted into the vein of the patient based on the pressure of the fluid associated with the second portion of the IV catheter.

Clause 10; The system of any of clauses 1-9, further comprising: a signal generator configured to transmit a signal that causes a pressure of a fluid associated with the vein of the patient to increase, wherein the at least one processor is further programmed or configured to: transmit a control signal to cause the signal generator to generate the signal that causes pressure of fluid associated with the vein of the patient to increase, and wherein, when receiving the data associated with the pressure of the fluid associated with the second portion of the IV catheter, the at least one processor is further programmed or configured to: receive the data associated with the pressure of the fluid associated with the second portion of the IV catheter based on transmitting the control signal to cause the signal generator to generate the signal that causes the pressure of fluid associated with the vein of the patient to increase.

Clause 11: The system of any of clauses 1-10, wherein, when determining whether the IV catheter is inserted into the vein of the patient based on the pressure of the fluid associated with the second portion of the IV catheter, the at least one processor is programmed or configured to: determine a pressure measurement detected by the pressure sensor based on the pressure of the fluid associated with the second portion of the IV catheter; determine that the pressure measurement detected by the pressure sensor satisfies a pressure measurement threshold, and determine whether the IV catheter is inserted into the vein of the patient based on determining that the pressure measurement detected by the pressure sensor satisfies the pressure measurement threshold.

Clause 12: The system of any of clauses 1-11, wherein the data associated with the pressure of the fluid disposed within the second portion of the IV catheter comprises: data associated with a first pressure of the fluid associated with the second portion of the IV catheter, and data associated with a second pressure of the fluid associated with the second portion of the IV catheter, and wherein, when determining whether the IV catheter is inserted into the vein of the patient based on the pressure of the fluid associated with the second portion of the IV catheter, the at least one processor is programmed or configured to: compare the first pressure of the fluid associated with the second portion of the IV catheter to the second pressure of the fluid associated with the IV catheter, and determine whether the first portion of the IV catheter is inserted into the vein of the patient based on comparing the first pressure of the fluid associated with the second portion IV catheter to the second pressure of the fluid associated with the second portion of the IV catheter.

Clause 13: The system of any of clauses 1-12, wherein, when comparing the first pressure of the fluid associated with the second portion of the IV catheter to the second pressure of the fluid associated with the second portion of the IV catheter, the at least one processor is programmed or configured to: determine a measured deviation between the first pressure of the fluid associated with the second portion IV catheter and the second pressure of the fluid associated with the IV catheter; compare the measured deviation to a permitted deviation threshold associated with placement of the IV catheter in the vein of the patient; and determine that the measured deviation satisfies the permitted deviation threshold, and wherein, when determining whether the first portion of the IV catheter is inserted into the vein of the patient based on comparing the first pressure of the fluid associated with the second portion of the IV catheter to the second pressure of the fluid associated with the second portion of the IV catheter, the at least one processor is programmed or configured to: determine whether the first portion of the IV catheter is inserted into the vein of the patient based on determining that the measured deviation satisfies the permitted deviation threshold.

Clause 14: The system of any of clauses 1-13, further comprising: a display configured to output an image indicating whether the IV catheter is inserted into the vein of the patient, wherein the at least one processor is further programmed or configured to: output, via the display, the image indicating whether the first portion of the IV catheter is inserted into the vein of the patient based on determining whether the first portion of the IV catheter is inserted into the vein of the patient.

Clause 15: A system, comprising: an intravenous (IV) catheter comprising a first portion configured to be inserted into a vein of a patient and a second portion configured to remain outside a patient when the first portion is inserted into the patient; wherein the second portion of the IV catheter comprises a signal generator configured to transmit a signal that causes a pressure of a fluid associated with the IV catheter to fluctuate.

Clause 16: The system of clause 15, wherein the signal generator is disposed within a lumen of the second portion of the IV catheter.

Clause 17: The system of clauses 15 or 16, wherein the signal generator is embedded in a sidewall of the second portion of the IV catheter

Clause 18: The system of any of clauses 15-17, wherein the signal generator is disposed along an exterior surface of the second portion of the IV catheter.

Clause 19: The system of any of clauses 15-18, wherein, when the first portion of the IV catheter is inserted into the vein of the patient and the second portion of the IV catheter is configured to be in fluid communication with the first portion of the IV catheter and the vein of the patient.

Clause 20: The system of any of clauses 15-19, wherein the signal that the signal generator is configured to transmit is configured to cause the pressure of the fluid in the vein of the patient to fluctuate when the first portion of the IV catheter is inserted into the vein of the patient.

Clause 21: The system of any of clauses 15-20, wherein the signal that the signal generator is configured to transmit is configured to forego causing the pressure of the fluid in the vein of the patient to fluctuate beyond a fluctuation threshold when the first portion of the IV catheter is not inserted into the vein of the patient.

Clause 22: The system of any of clauses 15-21, wherein the signal that the signal generator is configured to transmit is configured to forego causing the pressure of the fluid in the vein of the patient to fluctuate beyond a fluctuation threshold when a distal portion of the first portion of the IV catheter is not inserted into the vein of the patient.

Clause 23: The system of any of clauses 15-22, further comprising: a pressure sensor configured to detect a pressure of a fluid associated with the vein of the patient.

Clause 24: The system of any of clauses 15-23, wherein the pressure sensor is configured to detect the pressure of the fluid associated with the vein of the patient based on the pressure sensor being positioned along a proximal portion of a fluid path of the patient and the signal generator being placed along a distal portion of the fluid path of the patient.

Clause 25: The system of any of clauses 15-24, wherein the pressure sensor is configured to detect the pressure of the fluid associated with the vein of the patient based on the pressure sensor being positioned along a distal portion of a fluid path of the patient and the signal generator being placed along a proximal portion of the fluid path of the patient.

Clause 26: The system of any of clauses 15-25, wherein the pressure sensor is configured to detect the pressure of the fluid associated with the vein of the patient when the pressure sensor is disposed along a portion of the patient.

Clause 27: The system of any of clauses 15-26, wherein the pressure sensor is configured to detect the pressure of the fluid associated with the vein of the patient when the pressure sensor is positioned within a first predetermined distance from the vein of the patient and when the pressure sensor is positioned within a second predetermined distance from the signal generator.

Clause 28: The system of any of clauses 15-27, wherein the pressure sensor is configured to detect the pressure of the fluid associated with the vein of the patient when the pressure sensor is within a third predetermined distance to the signal generator along a fluid path between the pressure sensor and the signal generator.

Clause 29: The system of any of clauses 15-28, further comprising: at least one processor programmed or configured to; transmit a control signal to cause the signal generator to generate the signal that causes pressure of fluid included in the vein of the patient to fluctuate, receive data associated with the pressure of the fluid associated with the vein of the patient, and determine whether the first portion of the IV catheter is inserted into the vein of the patient based on the pressure of the fluid associated with the vein of the patient.

Clause 30: The system of any of clauses 15-29, further comprising: wherein, when receiving the data associated with the pressure of the fluid associated with the vein of the patient, the at least one processor is further programmed or configured to: receive the data associated with the pressure of the fluid associated with the vein of the patient based on transmitting the control signal to cause the signal generator to generate the signal that causes pressure of fluid associated with the vein of the patient to increase.

Clause 31: The system of any of clauses 15-30, wherein, when determining whether the IV catheter is inserted into the vein of the patient based on the pressure of the fluid associated with the vein of the patient, the at least one processor is programmed or configured to: determine a pressure measurement detected by a pressure sensor based on the pressure of the fluid associated with the vein of the patient; determine that the pressure measurement detected by the pressure sensor satisfies a pressure measurement threshold, and determine whether the first portion of the IV catheter is inserted into the vein of the patient based on determining that the pressure measurement detected by the pressure sensor satisfies the pressure measurement threshold.

Clause 32: The system of any of clauses 15-31, wherein the data associated with the pressure of the fluid associated with the vein of the patient comprises: data associated with a first pressure of the fluid associated with the vein of the patient, and data associated with a second pressure of the fluid associated with the vein of the patient, and wherein, when determining whether the IV catheter is inserted into the vein of the patient based on the pressure of the fluid associated with the vein of the patient, the at least one processor is programmed or configured to: compare the first pressure of the fluid associated with the vein of the patient to the second pressure of the fluid associated with the vein of the patient, and determine whether the IV catheter is inserted into the vein of the patient based on comparing the first pressure of the fluid associated with the vein of the patient to the second pressure of the fluid associated with the vein of the patient.

Clause 33: The system of any of clauses 15-33, wherein, when comparing the first pressure of the fluid associated with the vein of the patient to the second pressure of the fluid associated with the vein of the patient, the at least one processor is programmed or configured to: determine a measured deviation between the first pressure of the fluid associated with the vein of the patient and the second pressure of the fluid associated with the vein of the patient; compare the measured deviation to a permitted deviation threshold associated with placement of the IV catheter in the vein of the patient; and determine that the measured deviation satisfies the permitted deviation threshold, and wherein, when determining whether the IV catheter is inserted into the vein of the patient based on comparing the first pressure of the fluid associated with the vein of the patient to the second pressure of the fluid associated with the vein of the patient, the at least one processor is programmed or configured to: determining whether the IV catheter is inserted into the vein of the patient based on determining that the measured deviation satisfies the permitted deviation threshold.

Clause 34: The system of any of clauses 15-34, further comprising: a display configured to output an image indicating whether the IV catheter is inserted into the vein of the patient, wherein the at least one processor is further programmed or configured to: output, via the display, the image indicating whether the IV catheter is inserted into the vein of the patient based on determine whether the IV catheter is inserted into the vein of the patient.

Clause 35: A method, comprising: receiving, with at least one processor, data associated with a pressure of a fluid associated with a portion of an intravenous (IV) catheter; and determining, with at least one processor, whether the IV catheter is inserted into a vein of a patient based on the pressure of the fluid associated with the portion of the IV catheter.

Clause 36: The method of clause 35, wherein receiving the data associated with the pressure of the fluid associated with the portion of the IV catheter comprises: receiving data associated with a pressure of at least a portion of a fluid disposed in a fluid path extending from at least a portion of the vein of the patient to at least a first portion of the IV catheter or at least a second portion of the IV catheter.

Clause 37: The method of clauses 35 or 36, further comprising: transmitting a control signal to cause a signal generator to generate a signal that causes the pressure of the fluid associated with the IV catheter to fluctuate, wherein receiving the data associated with a pressure of a fluid associated with a portion of the IV catheter comprises: receiving the data associated with a pressure of a fluid associated with a portion of the IV catheter based on transmitting the control signal to cause the signal generator to generate the signal.

Clause 38: The method of any of clauses 35-37, wherein, determining whether the IV catheter is inserted into the vein of the patient based on the pressure of the fluid associated with the portion of the IV catheter comprises: determining that the pressure of the fluid associated with the portion of the IV catheter satisfies a pressure threshold, and determining whether the IV catheter is inserted into the vein of the patient based on determining that the pressure associated with the IV catheter satisfies the pressure threshold.

Clause 39: The method of any of clauses 35-38, wherein the data associated with the pressure of the fluid associated with the portion of the IV catheter comprises: data associated with a first pressure of the fluid associated with the IV catheter, and data associated with a second pressure of the fluid associated with the IV catheter, and wherein determining whether the IV catheter is inserted into the vein of the patient based on the pressure of the fluid associated with the IV catheter comprises: comparing the first pressure of the fluid associated with the IV catheter to the second pressure of the fluid associated with the IV catheter, and determining whether the IV catheter is inserted into the vein of the patient based on comparing the first pressure of the fluid associated with the IV catheter to the second pressure of the fluid associated with the IV catheter.

Clause 40: The method of any of clauses 35-39, wherein comparing the first pressure of the fluid associated with the IV catheter to the second pressure of the fluid associated with the IV catheter comprises; determining a measured deviation between the first pressure of the fluid associated with the IV catheter and the second pressure of the fluid associated with the IV catheter; comparing the measured deviation to a permitted deviation threshold associated with placement of the IV catheter in the vein of the patient; and determining that the measured deviation satisfies the permitted deviation threshold, and wherein determining whether the IV catheter is inserted into the vein of the patient based on comparing the first pressure of the fluid associated with the IV catheter to the second pressure of the fluid associated with the IV catheter comprises: determining whether the IV catheter is inserted into the vein of the patient based on determining that the measured deviation satisfies the permitted deviation threshold.

Clause 41: The method of any of clauses 35-40, further comprising: outputting an indication that the IV catheter is inserted into the vein of the patient based on determining whether the IV catheter is inserted into a vein of a patient.

Clause 42: A computer program product comprising at least one non-transitory computer-readable medium including one or more instructions that, when executed by at least one processor, cause the at least one processor to: receive data associated with a pressure of a fluid associated with a portion of an intravenous (IV) catheter; and determine whether the IV catheter is inserted into a vein of a patient based on the pressure of the fluid associated with the portion of the IV catheter.

Clause 43: The computer program product of clause 42, wherein the one or more instructions that cause the at least one processor to receive the data associated with the pressure of the fluid associated with the portion of the IV catheter cause the at least one processor to: receive data associated with a pressure of at least a portion of a fluid disposed in a fluid path extending from at least a portion of the vein of the patient to at least a first portion of the IV catheter or at least a second portion of the IV catheter.

Clause 44: The computer program product of clause 42 or 43, wherein the one or more instructions further cause the at least one processor to: transmit a control signal to cause a signal generator to generate a signal that causes the pressure of the fluid associated with the IV catheter to fluctuate, wherein the one or more instructions that cause the at least one processor to receive the data associated with a pressure of a fluid associated with a portion of the IV catheter cause the at least one processor to: receive the data associated with a pressure of a fluid associated with a portion of the IV catheter based on transmitting the control signal to cause the signal generator to generate the signal.

Clause 45: The computer program product of any of clauses 42-44, wherein the one or more instructions that cause the at least one processor to determine whether the IV catheter is inserted into the vein of the patient based on the pressure of the fluid associated with the portion of the IV catheter cause the at least one processor to: determine that the pressure of the fluid associated with the portion of the IV catheter satisfies a pressure threshold, and determine whether the IV catheter is inserted into the vein of the patient based on determining that the pressure associated with the IV catheter satisfies the pressure threshold.

Clause 46: The computer program product of any of clauses 42-45, wherein the data associated with the pressure of the fluid associated with the portion of the IV catheter comprises: data associated with a first pressure of the fluid associated with the IV catheter, and data associated with a second pressure of the fluid associated with the IV catheter, and wherein the one or more instructions that cause the at least one processor to determine whether the IV catheter is inserted into the vein of the patient based on the pressure of the fluid associated with the IV catheter cause the at least one processor to: compare the first pressure of the fluid associated with the IV catheter to the second pressure of the fluid associated with the IV catheter, and determine whether the IV catheter is inserted into the vein of the patient based on comparing the first pressure of the fluid associated with the IV catheter to the second pressure of the fluid associated with the IV catheter.

Clause 47: The computer program product of any of clauses 42-46, wherein the one or more instructions that cause the at least one processor to compare the first pressure of the fluid associated with the IV catheter to the second pressure of the fluid associated with the IV catheter cause the at least one processor to: determine a measured deviation between the first pressure of the fluid associated with the IV catheter and the second pressure of the fluid associated with the IV catheter; compare the measured deviation to a permitted deviation threshold associated with placement of the IV catheter in the vein of the patient; and determine that the measured deviation satisfies the permitted deviation threshold, and wherein the one or more instructions that cause the at least one processor to determine whether the IV catheter is inserted into the vein of the patient based on comparing the first pressure of the fluid associated with the IV catheter to the second pressure of the fluid associated with the IV catheter cause the at least one processor to: determine whether the IV catheter is inserted into the vein of the patient based on determining that the measured deviation satisfies the permitted deviation threshold.

Clause 48: The computer program product of any of clauses 42-47, wherein the one or more instructions further cause the at least one processor to: output an indication that the IV catheter is inserted into the vein of the patient based on determining whether the IV catheter is inserted into a vein of a patient.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of limits. As used in the specification and the claims, the singular form of “a,” “an,” and “the” include plural referents unless the context clearly dictates otherwise.

### BRIEF DESCRIPTION OF THE DRAWINGS

Additional advantages and details of embodiments or aspects of the present disclosure are explained in greater detail below with reference to the exemplary embodiments that are illustrated in the accompanying schematic figures, in which:

FIG. 1 is a diagram of non-limiting embodiments or aspects of an environment in which systems, devices, products, apparatus, and/or methods, described herein, may be implemented;

FIG. 2 is a diagram of non-limiting embodiments or aspects of components of one or more devices and/or one or more systems of FIG. 1;

FIG. 3 is a flowchart of a non-limiting embodiment or aspects of a process for determining whether a catheter is inserted into a vein of a patient:

FIG. 4A is a perspective view of an implementation of non-limiting embodiments or aspects of a system for determining whether a catheter is inserted into a vein of a patient;

FIG. 4B is a perspective view of an implementation of non-limiting embodiments or aspects of a system for determining whether a catheter is inserted into a vein of a patient;

FIG. 4C is a perspective view of an implementation of non-limiting embodiments or aspects of a system for determining whether a catheter is inserted into a vein of a patient;

FIG. 4D is a perspective view of an implementation of non-limiting embodiments or aspects of a system for determining whether a catheter is inserted into a vein of a patient;

FIG. 4E is a perspective view of an implementation of non-limiting embodiments or aspects of a system for determining whether a catheter is inserted into a vein of a patient;

FIG. 4F is a perspective view of an implementation of non-limiting embodiments or aspects of a system for determining whether a catheter is inserted into a vein of a patient;

FIG. 5A is a perspective view of an implementation of non-limiting embodiments or aspects of a system for determining whether a catheter is inserted into a vein of a patient;

FIG. 5B is a perspective view of an implementation of non-limiting embodiments or aspects of a system for determining whether a catheter is inserted into a vein of a patient;

FIG. 5C is a perspective view of an implementation of non-limiting embodiments or aspects of a system for determining whether a catheter is inserted into a vein of a patient;

FIG. 5D is a perspective view of an implementation of non-limiting embodiments or aspects of a system for determining whether a catheter is inserted into a vein of a patient;

FIG. 5E is a perspective view of an implementation of non-limiting embodiments or aspects of a system for determining whether a catheter is inserted into a vein of a patient;

FIG. 5F is a perspective view of an implementation of non-limiting embodiments or aspects of a system for determining whether a catheter is inserted into a vein of a patient;

FIG. 6A is a perspective view of an implementation of non-limiting embodiments or aspects of a system for determining whether a catheter is inserted into a vein of a patient;

FIG. 6B is a perspective view of an implementation of non-limiting embodiments or aspects of a system for determining whether a catheter is inserted into a vein of a patient;

FIG. 6C is a perspective view of an implementation of non-limiting embodiments or aspects of a system for determining whether a catheter is inserted into a vein of a patient;

FIG. 6D is a perspective view of an implementation of non-limiting embodiments or aspects of a system for determining whether a catheter is inserted into a vein of a patient;

FIG. 6E is a perspective view of an implementation of non-limiting embodiments or aspects of a system for determining whether a catheter is inserted into a vein of a patient;

FIG. 6F is a perspective view of an implementation of non-limiting embodiments or aspects of a system for determining whether a catheter is inserted into a vein of a patient;

FIG. 7A is a perspective view of an implementation of non-limiting embodiments or aspects of a system for determining whether a catheter is inserted into a vein of a patient;

FIG. 78 is a perspective view of an implementation of non-limiting embodiments or aspects of a system for determining whether a catheter is inserted into a vein of a patient;

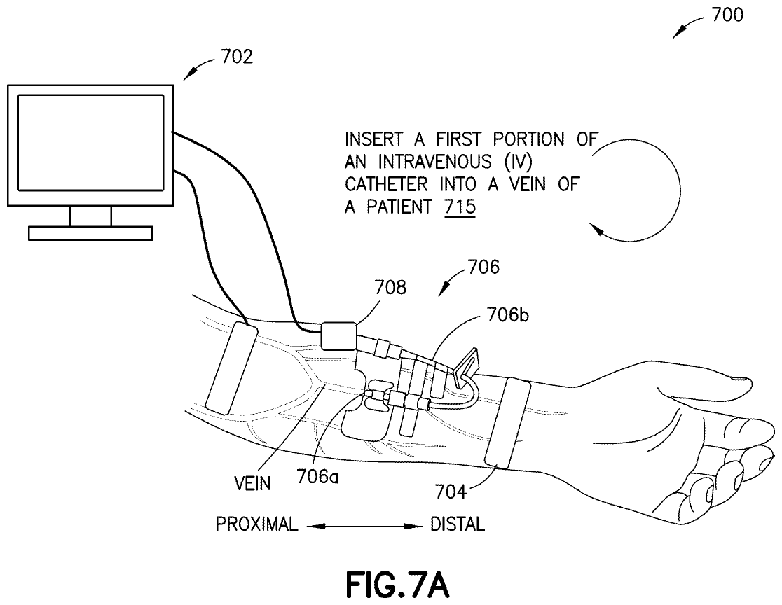

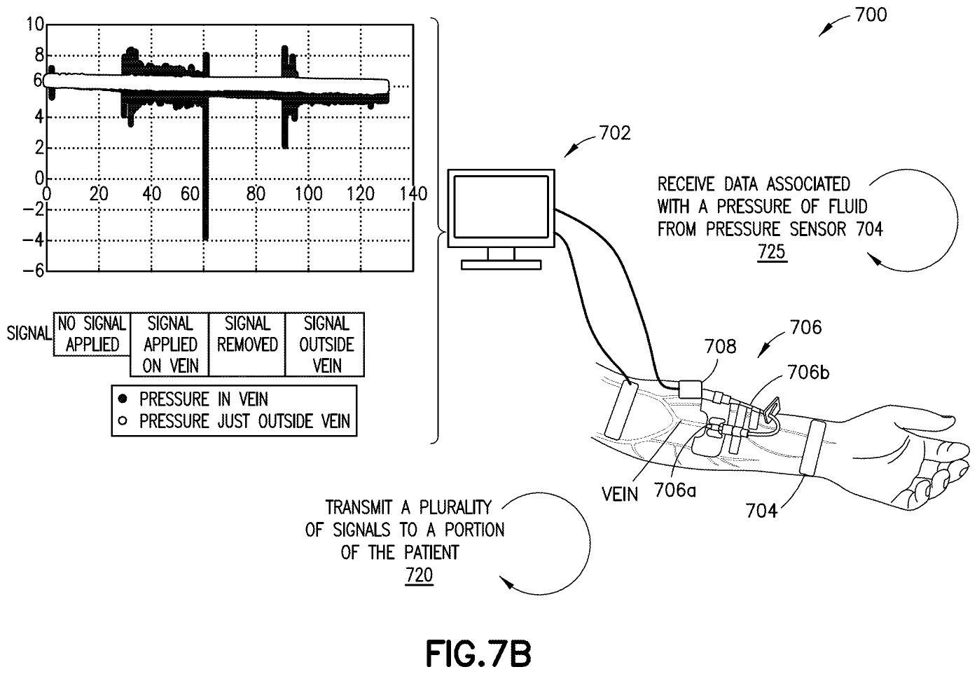

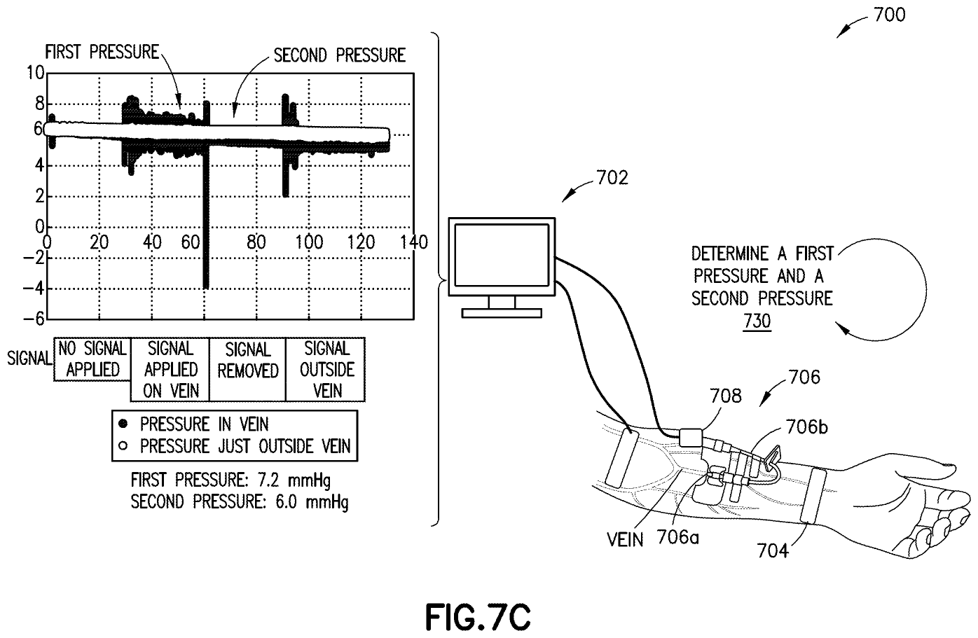

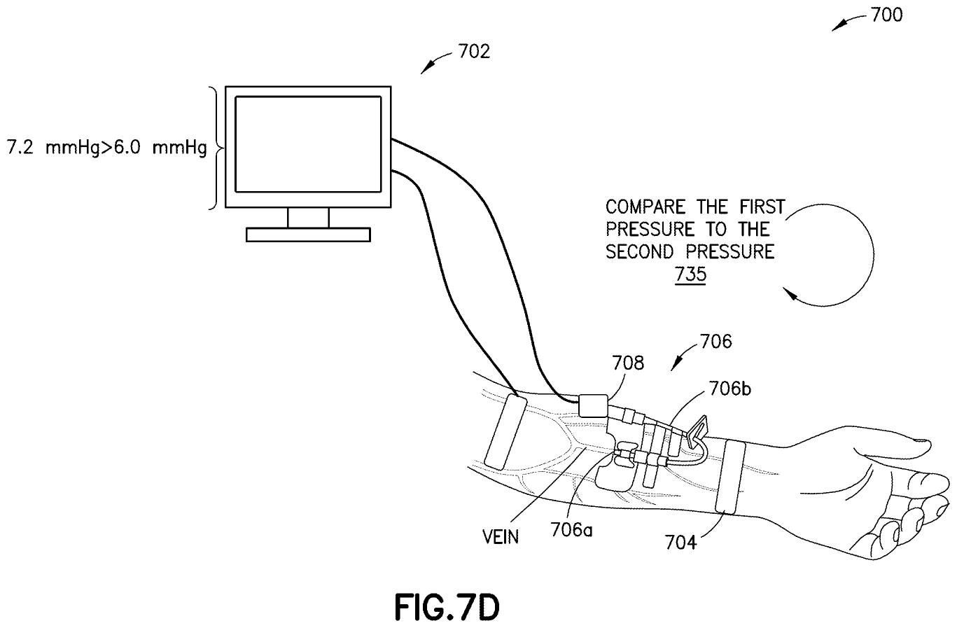

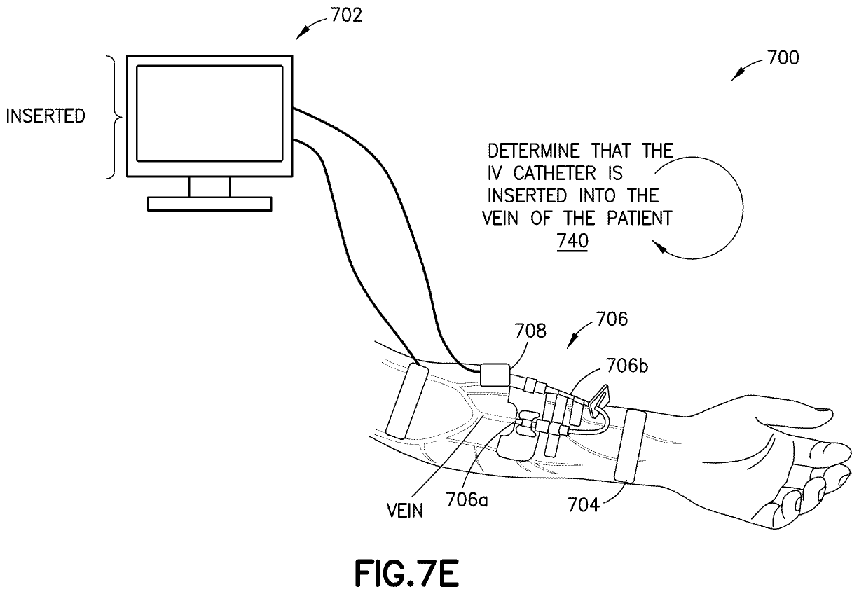

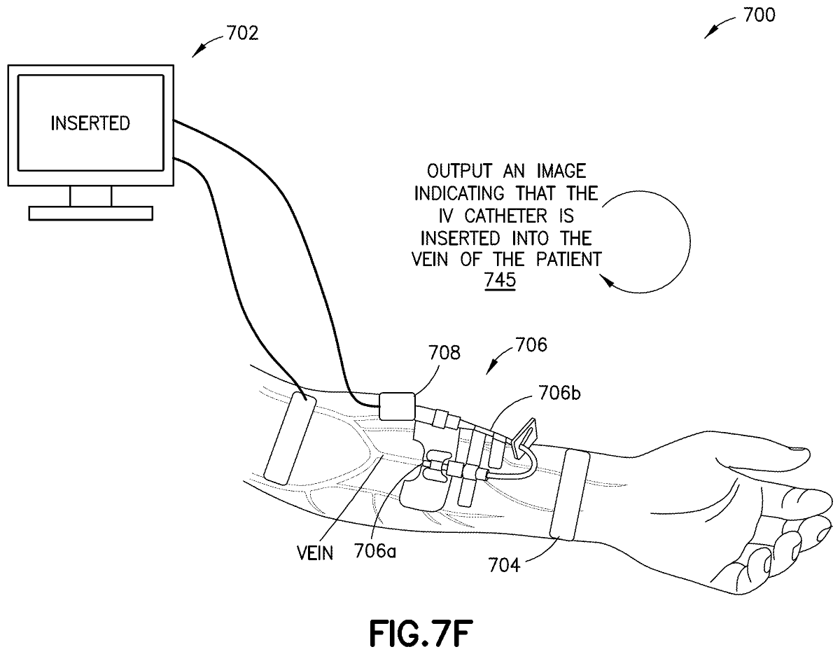

FIG. 7C is a perspective view of an implementation of non-limiting embodiments or aspects of a system for determining whether a catheter is inserted into a vein of a patient;

FIG. 7D is a perspective view of an implementation of non-limiting embodiments or aspects of a system for determining whether a catheter is inserted into a vein of a patient;

FIG. 7E is a perspective view of an implementation of non-limiting embodiments or aspects of a system for determining whether a catheter is inserted into a vein of a patient; and

FIG. 7F is a perspective view of an implementation of non-limiting embodiments or aspects of a system for determining whether a catheter is inserted into a vein of a patient.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” lateral,” “longitudinal,” and derivatives thereof shall relate to the present disclosure as it is oriented in the drawing figures. However, it is to be understood that the present disclosure may assume various alternative variations and step sequences, except where expressly specified to the contrary. It is also to be understood that the specific devices and processes illustrated in the attached drawings, and described in the following specification, are simply exemplary embodiments or aspects of the present disclosure. Hence, specific dimensions and other physical characteristics related to the embodiments or aspects of the embodiments disclosed herein are not to be considered as limiting unless otherwise indicated.

As used herein, proximal shall refer to a part or direction towards or located nearest to an individual, while distal shall refer to a part or direction located away or furthest from an individual, Reference to a patient may be to any being, human or animal. Reference to a clinician may be to any person or thing giving treatment, e.g., a nurse, doctor, machine intelligence, caregiver, or even self-treatment.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least in partially on” unless explicitly stated otherwise.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit (e.g., a third unit located between the first unit and the second unit) processes data from one unit and transmits processed data to the second unit. It will be appreciated that numerous other arrangements are possible.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, and/or the like.

As used herein, the terms “client” and “client device” may refer to one or more devices, such as processors, storage devices, and/or similar computer components, which access a service made available by a server. In some non-limiting embodiments or aspects, a client device may include an electronic device configured to communicate with one or more networks such as one or more desktop computers, one or more tablet computers, one or more mobile devices (e.g., cellular phones, smartphones, PDAs, wearable devices, such as watches, glasses, lenses, and/or clothing, and/or the like), and/or the like.

As used herein, the term “server” may refer to one or more computing devices, such as processors, storage devices, and/or similar computer components that communicate with client devices and/or other computing devices over a network, such as the Internet or private networks and, in some examples, facilitate communication among other servers and/or client devices.

As used herein, the term “system” may refer to one or more devices or combinations of devices such as, but not limited to, processors, servers, client devices, software applications, and/or other like components. In addition, reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

As used herein, the term “fluid path” may refer to a path along which fluid may flow within one or more channels established by walls of one or more structures (e.g., a vein, a catheter, a catheter inserted into a vein, and/or the like).

Non-limiting embodiments of the present disclosure are directed to systems, methods, and computer program products for determining whether a catheter is inserted into a vein of a patient. In one embodiment, a system may include an IV catheter, the IV catheter including a first portion configured to be inserted into a vein of a patient, and a second portion configured to remain outside a patient when the first portion is inserted into the patient. The second portion of the IV catheter may include a pressure sensor configured to detect a pressure of a fluid associated with the second portion of the V catheter. In embodiments or aspects, the system may include at least one processor programmed or configured to receive data associated with the pressure of the fluid associated with the second portion of the IV catheter, and determine whether the IV catheter is inserted into the vein of the patient based on the pressure of the fluid associated with the second portion of the IV catheter. In some non-limiting embodiments or aspects, the system may further provide an indication to clinicians as to whether the IV catheter is inserted into the vein of the patient.

In this way, non-limiting embodiments or aspects of the present disclosure enable a clinician to determine whether a portion (e.g., a tip) of a catheter is dislodged from a vein of the patient that the portion of the catheter is being and/or was inserted into (e.g., during initial placement of the portion of the catheter that was inserted, during subsequent placement of the portion of the catheter that was inserted, and/or the like). Additionally, non-limiting embodiments or aspects of the present disclosure enable clinicians to determine whether the portion of the catheter is dislodged from the vein of the patient when the patients are unable to notify a clinician about pain associated with IV infiltration such as, for example, where the patients are unconscious, younger (e.g., infants, toddlers, and/or the like), and/or the like. The clinicians may then be able to stop the infusion of fluids into the patient and insert a new catheter properly (e.g., such that the catheter is inserted into the vein and/or a different vein) of the patient based on an indication of whether the IV catheter is inserted into the vein of the patient. Additionally, by virtue of the non-limiting embodiments described herein, reduction of false indications of a dislodged catheter due to noise (e.g., noise generated by pumping of a patient's heart, breathing, movement by the patient, and/or the like) may be realized.

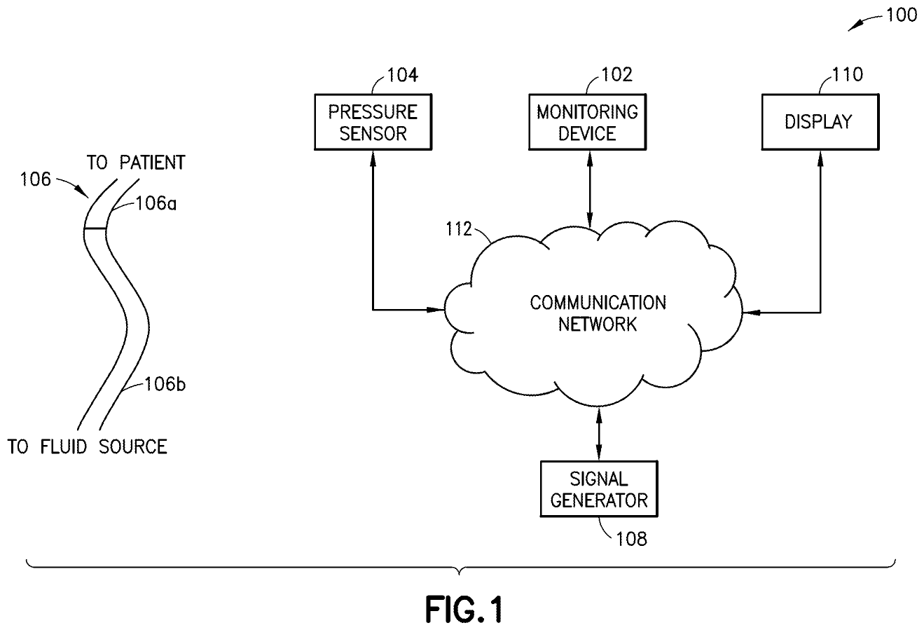

Referring now to FIG. 1, FIG. 1 is a diagram of an example environment 100 in which devices, systems, methods, and/or products described herein may be implemented. As shown in FIG. 1, environment 100 may include monitoring device 102, pressure sensor 104, intravenous (IV) catheter 106, signal generator 108, display 110, and/or communication network 112. In some non-limiting embodiments or aspects, IV catheter 106 may include a first portion 106a and a second portion 106b.

Monitoring device 102 may include one or more devices configured to be in communication with pressure sensor 104, signal generator 108, and/or display 110 via communication network 112, For example, monitoring device 102 may include a computing device (e.g., a laptop computer, a desktop computer, a server, a mobile device, and/or the like), In some non-limiting embodiments or aspects, monitoring device 102 may be configured to communicate via a short-range wireless communication connection (e.g., a near-field communication (NFC) connection, a radio-frequency identification (RFID) communication connection, a Bluetooth® communication connection, and/or the like). In some non-limiting embodiments or aspects, monitoring device 102 may be associated with a clinician as described herein. In some non-limiting embodiments or aspects, monitoring device 102 may be included in one or more systems such as, for example, an infusion system for delivering infusions of fluids and/or drugs via an IV catheter (e.g., IV catheter 106), via a device (e.g., a mobile device, a tablet, a personal computer, a cloud-based computing system, and/or the like) in communication with one or more components (e.g., one or more components associated with an IV catheter, a needless connector, a different connector or port, a pump, a clamp, and/or the like), and/or the like. Additionally, or alternatively, monitoring device 102 may be configured to be in communication with other monitoring devices (e.g., a patient monitor, a bed monitor, a non-invasive blood pressure (NIBP) monitor, an oxygen saturation (SpO2) monitor, an invasive blood pressure (IBP) monitor, an electrocardiogram (EGG or EKG) and/or the like.

Pressure sensor 104 may include one or more devices configured to be in communication with monitoring device 102, signal generator 108, and/or display 110. For example, pressure sensor 104 may include a device configured to measure a pulse wave transmitted through fluid via ultrasound waves, physical pressure (e.g., a piston), auditory waves, mechanical vibrations, light and/or optic waves, magnetic waves, radio waves, and/or the like. Additionally, or alternatively, pressure sensor 104 may include a device configured to measure a temperature of tissue (e.g., tissue associated with a fluid path). In some non-limiting embodiments or aspects, pressure sensor 104 may include a piezoelectric sensor, a piezoelectric transducer, and/or the like. In some non-limiting embodiments or aspects, pressure sensor 104 may be included in a generalized housing, such as a stethoscope or other medical practitioner-worn device, which may be temporarily provided in proximity to the target patient.

In some non-limiting embodiments or aspects, pressure sensor 104 may be configured to communicate via a short-range wireless communication connection. In some non-limiting embodiments or aspects, pressure sensor 104 may be associated with a clinician as described herein. In some non-limiting embodiments or aspects, pressure sensor 104 may be configured to detect a pressure of a fluid. For example, pressure sensor 104 may be configured to detect a pressure of fluid disposed within the first portion 106a of IV catheter 106, the second portion 106b of IV catheter 106, and/or a vein of a patient. In some non-limiting embodiments or aspects, pressure sensor 104 may be configured to detect the pressure of fluid included in a fluid path. For example, pressure sensor 104 may be configured to detect the pressure of fluid disposed within the first portion 106a of IV catheter 106, the second portion 106b of IV catheter 106, and/or the vein of the patient having the first portion 106a of IV catheter 106 inserted therein, where the fluid path includes at least a portion of the fluid disposed within the first portion 106a of IV catheter 106, the second portion 106b of the IV catheter 106, and/or the vein of the patient. In some non-limiting embodiments or aspects, pressure sensor 104 may be configured to detect fluctuations in the pressure of the fluid disposed within the first portion 106a of IV catheter 106, the second portion 106b of IV catheter 106, and/or the vein of the patient having the first portion 106a of IV catheter 106 inserted therein based on generation of one or more signals by signal generator 108, described herein.

In some non-limiting embodiments or aspects, pressure sensor 104 may be configured to be disposed along an exterior of a patient. For example, pressure sensor 104 may be configured to be disposed along an exterior of a patient (e.g., included in a band positioned along the patient, included in a blood pressure cuff positioned along the patient, included in a catheter dressing in contact with the first portion 106a and/or the second portion 106b of IV catheter 106, and/or the like) and pressure sensor 104 may be configured to detect a pressure of a fluid associated with a vein of a patient when disposed along the exterior of the patient. In such an example, the vein of the patient may be a vein which a first portion of an IV catheter (e.g., a first portion 106a of IV catheter 106) is inserted into.

IV catheter 106 may include one or more catheters configured to be in fluid communication with a vein of a patient. For example, IV catheter 106 may include a catheter such as, for example, a peripheral venous catheter, a central venous catheter, a midline catheter, and/or the like. In some non-limiting embodiments or aspects, IV catheter 106 may include a first portion 106a and a second portion 106b. For example, the first portion 106a of IV catheter 106 may be configured to be inserted into a vein of a patient (e.g., may be sub-dermal). In an example, the second portion 106b of IV catheter 106 may be in fluid communication with the first portion 106a of IV catheter 106. In such an example, the second portion 106b of IV catheter 106 may be configured to be in fluid communication with the first portion 106a of IV catheter 106, In some non-limiting embodiments or aspects, the second portion 106b of IV catheter 106 may be configured to remain outside (e.g., not sub-dermal) the patient when the first portion 106a of IV catheter 106 is inserted into a patient (e.g, into a vein of the patient, into tissue of the patient, and/or the like) In some non-limiting embodiments or aspects, the first portion 106a and/or the second portion 106b of IV catheter 106 may include pressure sensor 104. For example, the first portion 106a and/or the second portion 106b of IV catheter 106 may include pressure sensor 104 disposed within a lumen of the first portion 106a of IV catheter 106 and/or the second portion 106b of IV catheter 106 thereof. In another example, the first portion 106a and/or the second portion 106b of IV catheter 106 may include pressure sensor 104 embedded in a side wall of the first portion 106a of IV catheter 106 and/or the second portion 106b of IV catheter 106 therein. In another example, the first portion 106a and/or the second portion 106b of IV catheter 106 may include pressure sensor 104 disposed along an exterior surface of the first portion 106a of IV catheter 106 and/or the second portion 106b of IV catheter 106 thereof. In some non-limiting embodiments or aspects, the first portion 106a and/or the second portion 106b of IV catheter 106 may include signal generator 108, as described herein. For example, the first portion 106a and/or the second portion 106b of IV catheter 106 may include signal generator 108 disposed within a lumen of the first portion 106a of IV catheter 106 and/or the second portion 106b of IV catheter 106 thereof. In another example, the first portion 106a and/or the second portion 106b of IV catheter 106 may include signal generator 108 embedded in a side wall of the first portion 106a of IV catheter 106 and/or the second portion 106b of IV catheter 106 therein. In another example, the first portion 106a and/or the second portion 106b of IV catheter 106 may include signal generator 108 disposed along an exterior surface of the first portion 106a of IV catheter 106 and/or the second portion 106b of IV catheter 106 thereof.

Signal generator 108 may include one or more devices configured to be in communication with monitoring device 102, pressure sensor 104, and/or display 110.

For example, signal generator 108 may include a device configured to generate a pulse wave via ultrasound waves, physical pressure (e.g., a piston), auditory waves, mechanical vibrations, light and/or optic waves, magnetic waves, radio waves, and/or the like. In some non-limiting embodiments or aspects, signal generator 108 may include a solenoid, a squeezing cuff (e.g., air inflation or other cuff with rapid pulsatile occlusion), a direct current (DC) motor, a mechanical pressure signal generator, and/or the like, Additionally, or alternatively, pressure sensor 104 may include a device configured to generate and/or remove heat from tissue (e.g., tissue associated with a fluid path), In some non-limiting embodiments or aspects, signal generator 108 may be included in a stethoscope. In some non-limiting embodiments or aspects, signal generator 108 may be configured to communicate via a short-range wireless communication connection. In some non-limiting embodiments or aspects, signal generator 108 may be associated with a clinician as described herein. In some non-limiting embodiments or aspects, signal generator 108 may be configured to generate one or more signals (e.g, one or more controlled and/or known signals) to cause a pressure of fluid in a fluid path to fluctuate. For example, signal generator 108 may be configured to generate one or more signals to cause a pressure of fluid in a fluid path to increase, decrease, and/or the like.

In some non-limiting embodiments or aspects, signal generator 108 may be configured to be disposed along an exterior of a patient. For example, signal generator 108 may be configured to be disposed along an exterior of a patient (e.g., included in a band positioned along the patient, included in a blood pressure cuff positioned along the patient, included in a catheter dressing in contact with the first portion 106a and/or the second portion 106b of IV catheter 106, and/or the like) and signal generator 108 may be configured to generate one or more signals to cause a pressure of a fluid associated with a vein of a patient to fluctuate when disposed along the exterior of the patient. In such an example, the vein of the patient may be a vein which a first portion of an IV catheter (e.g., a first portion 106a of IV catheter 106) is inserted into.

Communication network 112 may include one or more wired and/or wireless networks. For example, communication network 112 may include a cellular network (e.g., a long-term evolution (LTE) network, a third generation (3G) network, a fourth generation (4G) network, a fifth generation network (5G) network, a code division multiple access (CDMA) network, and/or the like), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of these or other types of networks.

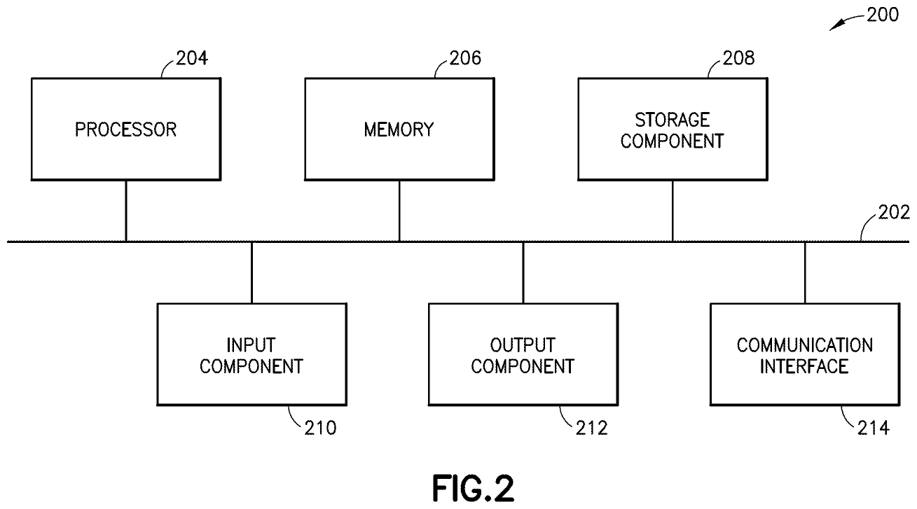

Referring now to FIG. 2, FIG. 2 is a diagram of example components of a device 200. Device 200 may correspond to monitoring device 102, pressure sensor 104, signal generator 108, and/or display 110. In some non-limiting embodiments or aspects, monitoring device 102, pressure sensor 104, signal generator 108, and/or display 110 may include at least one device 200 and/or at least one component of device 200. As shown in FIG. 2, device 200 may include bus 202, processor 204, memory 206, storage component 208, input component 210, output component 212, and/or communication interface 214.

Bus 202 may include a component that permits communication among the components of device 200, In some non-limiting embodiments or aspects, processor 204 may be implemented in hardware, firmware, or a combination of hardware and software. For example, processor 204 may include a processor (e.g. a central processing unit (CPU), a graphics processing unit (GPU), an accelerated processing unit (APU), and/or the like), a microprocessor, a digital signal processor (DSP), and/or any processing component (e.g., a field-programmable gate array (FPGA), an application-specific integrated circuit (ASIC), and/or the like), and/or the like, which can be programmed to perform a function. Memory 206 may include a random-access memory (RAM), a read-only memory (ROM), and/or another type of dynamic or static storage device (e.g., a flash memory, a magnetic memory, an optical memory, and/or the like) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, and/or the like), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touch screen display, a keyboard, a keypad, a mouse, a button, a switch, a microphone, and/or the like). Additionally, or alternatively, input component 210 may include a sensor for sensing information (e.g., a global positioning system (GPS) component, an accelerometer, a gyroscope, an actuator, an NFC sensor, an RFID sensor, an optical sensor, a bar code reader, and/or the like). Output component 212 may include a component that provides output information from device 200 (e.g., a display, a speaker, one or more light-emitting diodes (LEDs), and/or the like).

Communication interface 214 may include a transceiver-like component (e.g., a transceiver, a separate receiver and transmission source, and/or the like) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally, or alternatively, hardwired circuitry may be used in place of or in combination with software instructions to perform one or more processes described herein, Thus, embodiments or aspects described herein are not limited to any specific combination of hardware circuitry and software.

Memory 206 and/or storage component 208 may include data storage or one or more data structures (e.g., a database, and/or the like). Device 200 may be capable of receiving information from, storing information in, communicating information to, or searching information stored in the data storage or one or more data structures in memory 206 and/or storage component 208.

The number and arrangement of components shown in FIG. 2 are provided as an example. In some non-limiting embodiments or aspects, device 200 may include additional components, fewer components, different components, or differently arranged components than those shown in FIG. 2. Additionally, or alternatively, a set of components (e.g., one or more components) of device 200 may perform one or more functions described as being performed by another set of components of device 200.

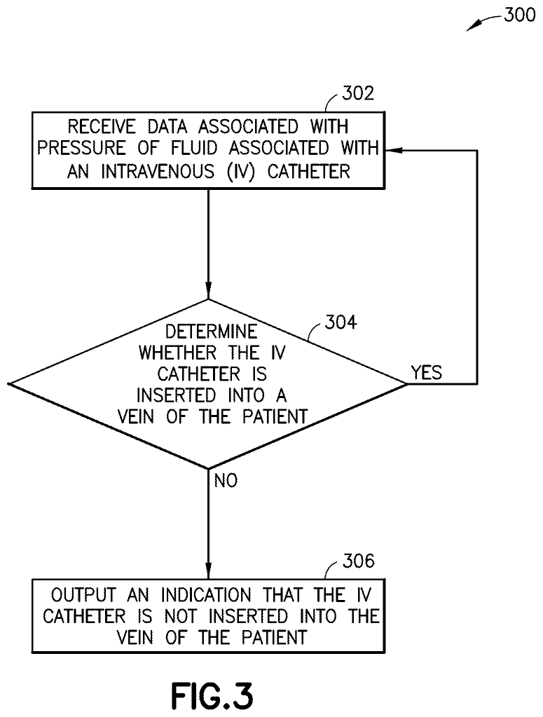

Referring now to FIG. 3, FIG. 3 is a flowchart of a non-limiting embodiment or aspects of a process 300 for determining whether a catheter is inserted into a vein of a patient. In some non-limiting embodiments or aspects, one or more of the functions described with respect to process 300 may be performed (e.g., completely, partially, and/or the like) by monitoring device 102. In some non-limiting embodiments or aspects, one or more of the steps of process 300 may be performed (e.g., completely, partially, and/or the like) by another device or a group of devices separate from and/or including monitoring device 102, such as pressure sensor 104, signal generator 108, and/or display 110.

As shown in FIG. 3, at step 302, process 300 may include receiving data associated with a pressure of fluid associated with an IV catheter. For example, monitoring device 102 may receive data associated with a pressure of a fluid associated with IV catheter 106, The fluid associated with IV catheter 106 may include fluid that is included in a fluid path involving IV catheter 106. In some non-limiting embodiments or aspects, monitoring device 102 may receive the data associated with the pressure of the fluid associated with IV catheter 106 at one or more points in time. For example, monitoring device 102 may receive the data associated with the pressure of the fluid associated with IV catheter 106 periodically (e.g., at a point in time, at a plurality of points in time, at a plurality of points in time associated with one or more time intervals, and/or the like) and/or continuously. In some non-limiting embodiments or aspects, monitoring device 102 may receive the data associated with the pressure of the fluid associated with IV catheter 106 from pressure sensor 104. For example, monitoring device 102 may receive the data associated with the pressure of the fluid associated with IV catheter 106 from pressure sensor 104 based on pressure sensor 104 being included in IV catheter 106 (e.g., included in a first portion 106a of IV catheter 106 and/or a second portion 106b of IV catheter 106) and/or disposed along a patient having IV catheter 106 inserted (e.g., inserted into a vein of the patient, partially inserted into the vein of the patient, and/or inserted into tissue of the patient different from the vein of the patient) therein.

In some non-limiting embodiments or aspects, monitoring device 102 may receive data associated with a pressure of a fluid associated with IV catheter 106 based on placement of IV catheter 106 along a fluid path. For example, monitoring device 102 may receive data associated with a pressure of a fluid associated with IV catheter 106 based on placement of IV catheter 106 along a fluid path by a clinician. In such an example, the clinician may place IV catheter 106 along the fluid path by inserting a first portion 106a of IV catheter 106 into a vein of a patient to establish the fluid path, the fluid path extending from at least a portion of the vein of the patient to at least a portion of IV catheter 106. In some non-limiting embodiments or aspects, pressure sensor 104 may measure the pressure of the fluid associated with IV catheter 106 and pressure sensor 104 may transmit data associated with the pressure of the fluid associated with IV catheter 106 to monitoring device 102. For example, pressure sensor 104 may measure the pressure of fluid included in the first portion 106a of IV catheter 106, the second portion 106b of IV catheter 106, and/or a vein of a patient that the first portion 106a of IV catheter 106 is inserted into and pressure sensor 104 may transmit data associated with the pressure of the fluid to monitoring device 102.

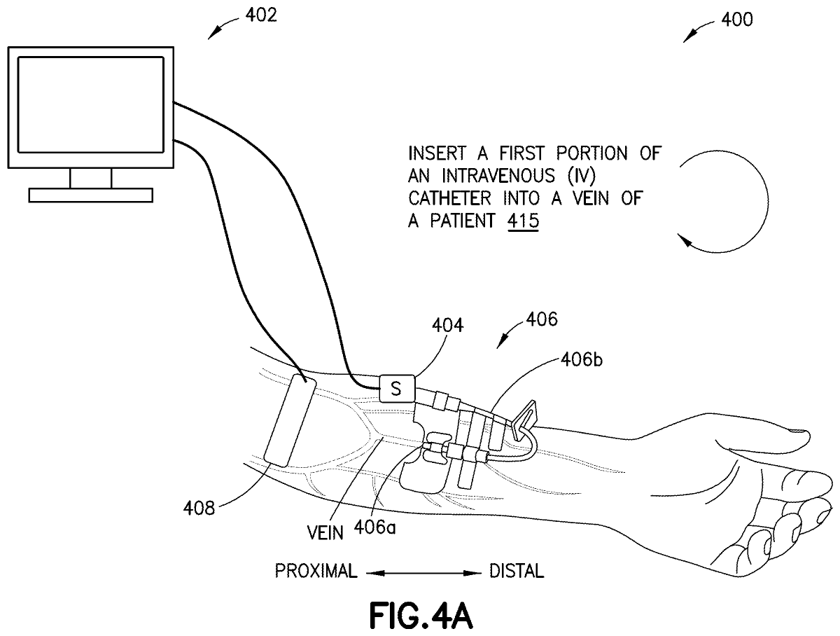

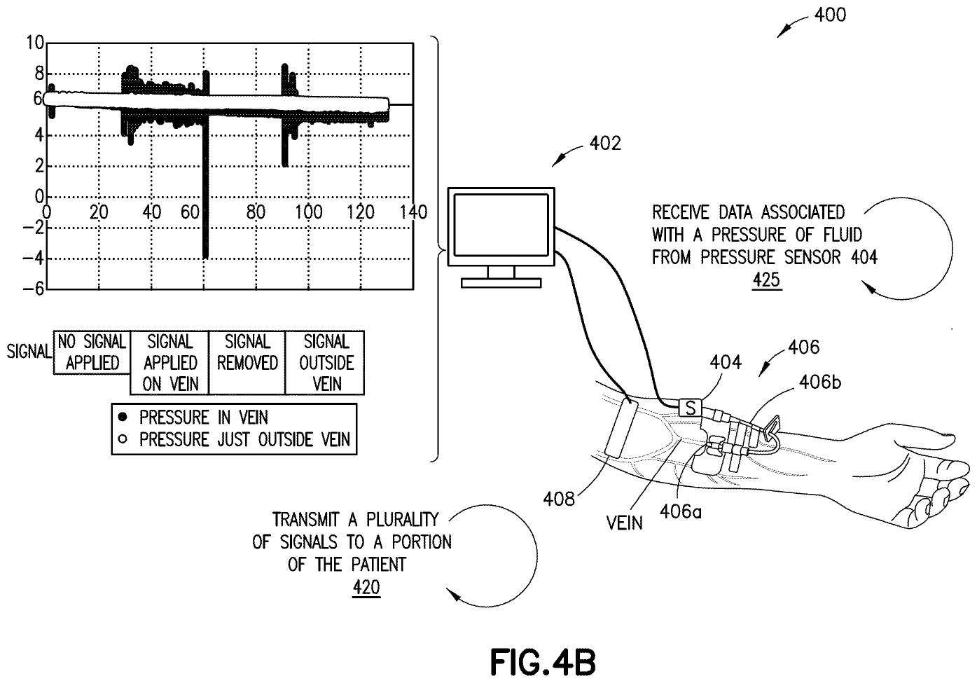

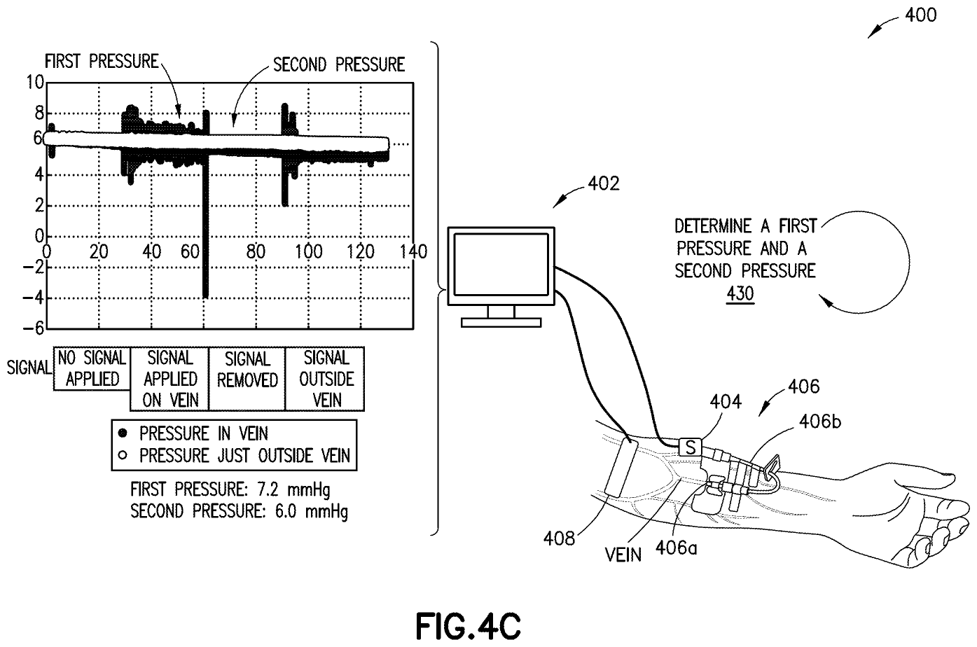

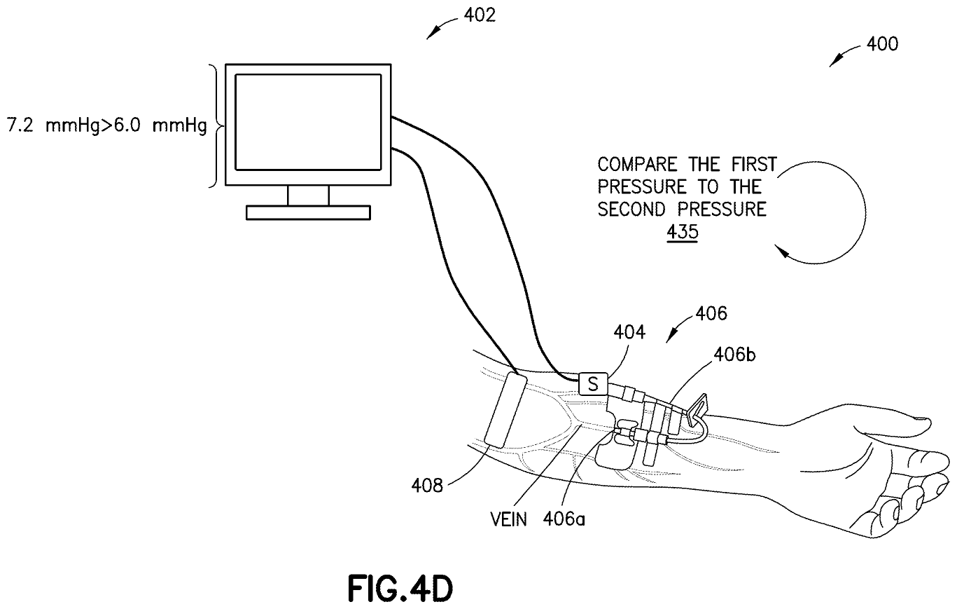

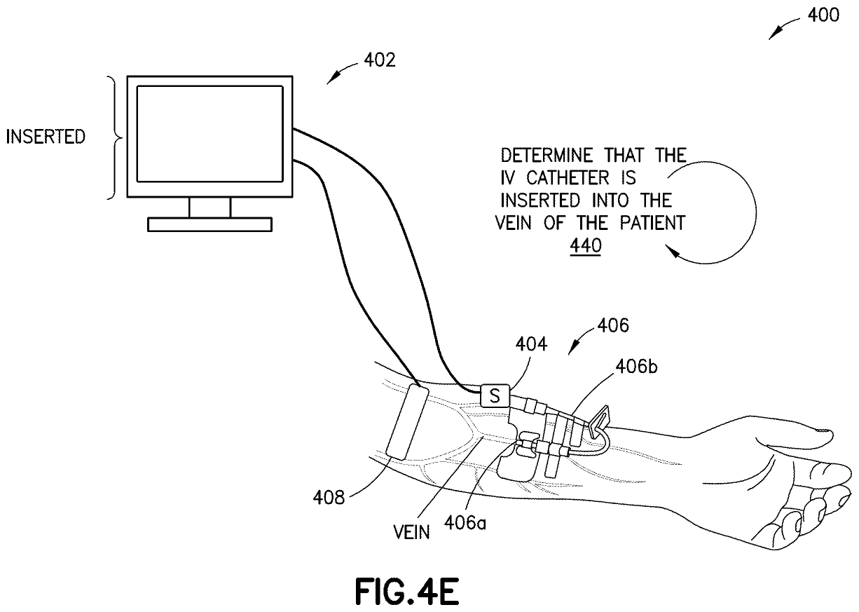

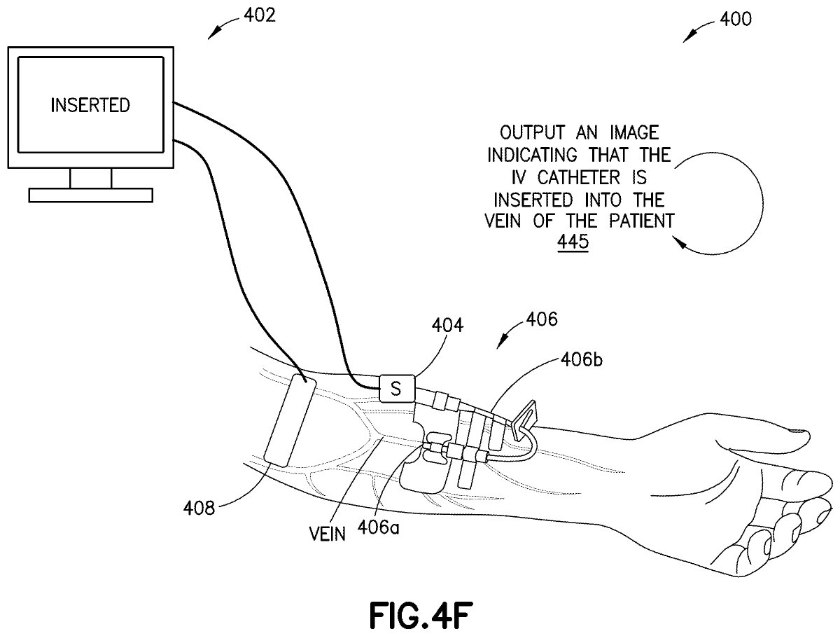

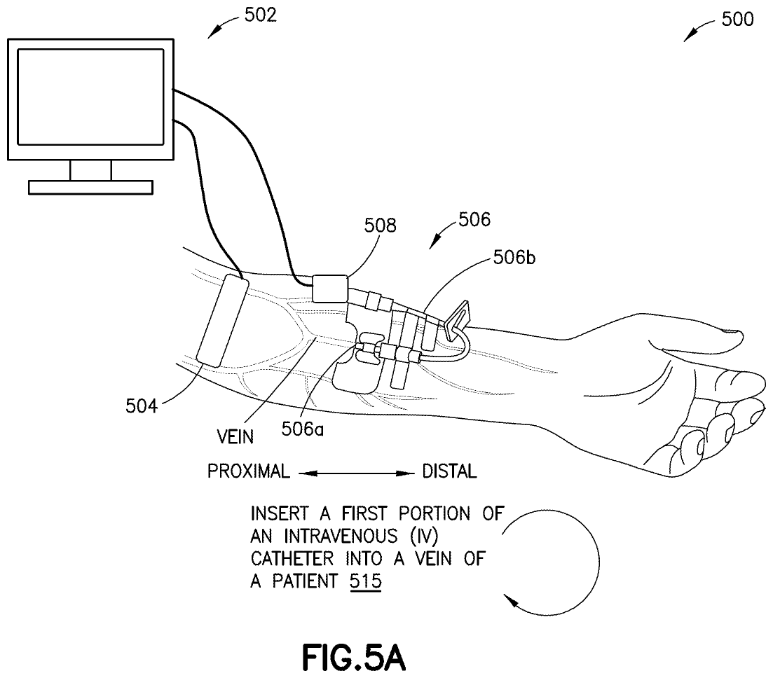

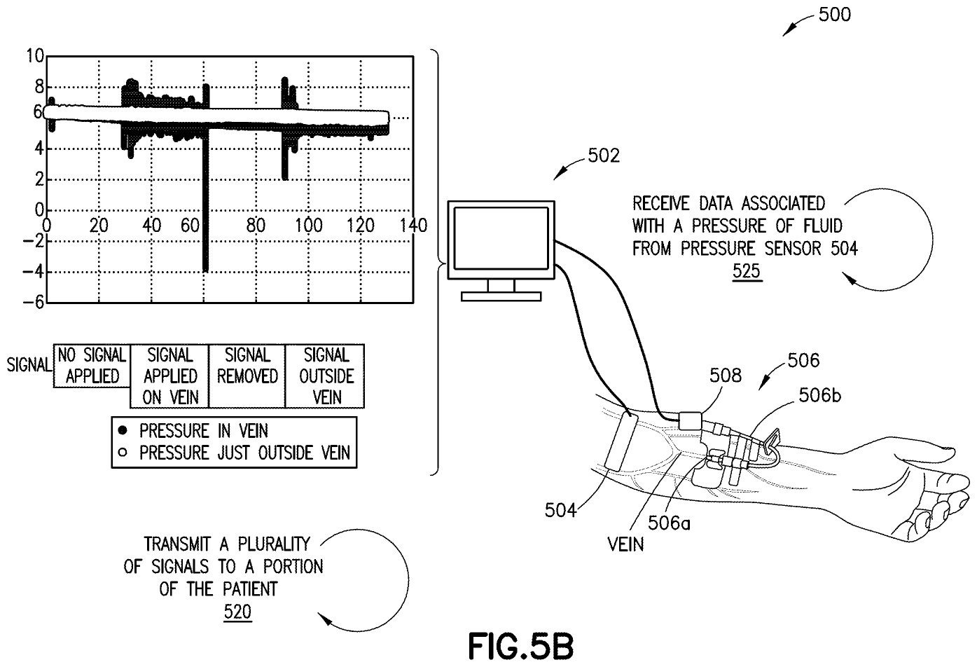

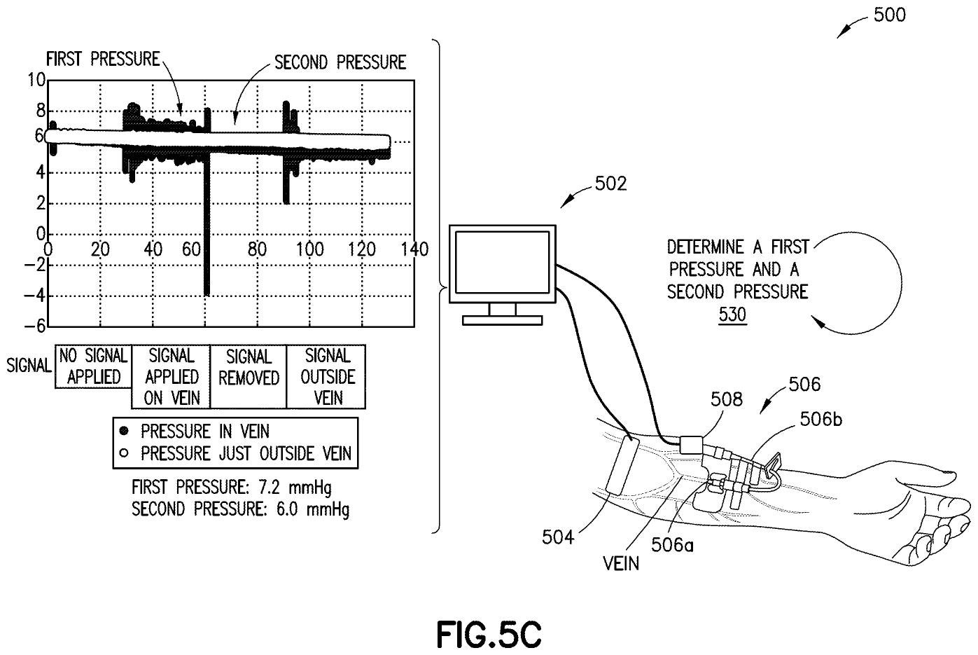

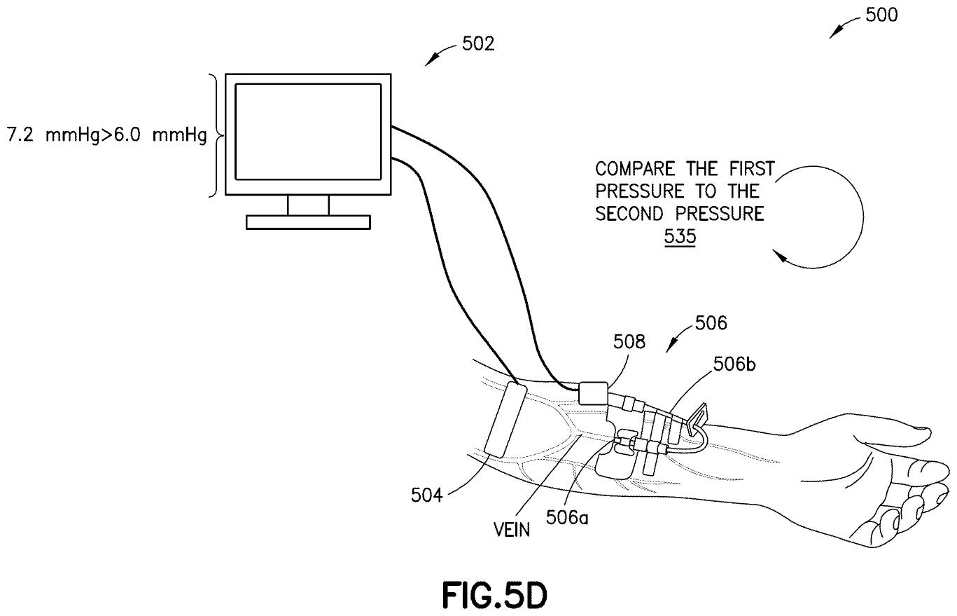

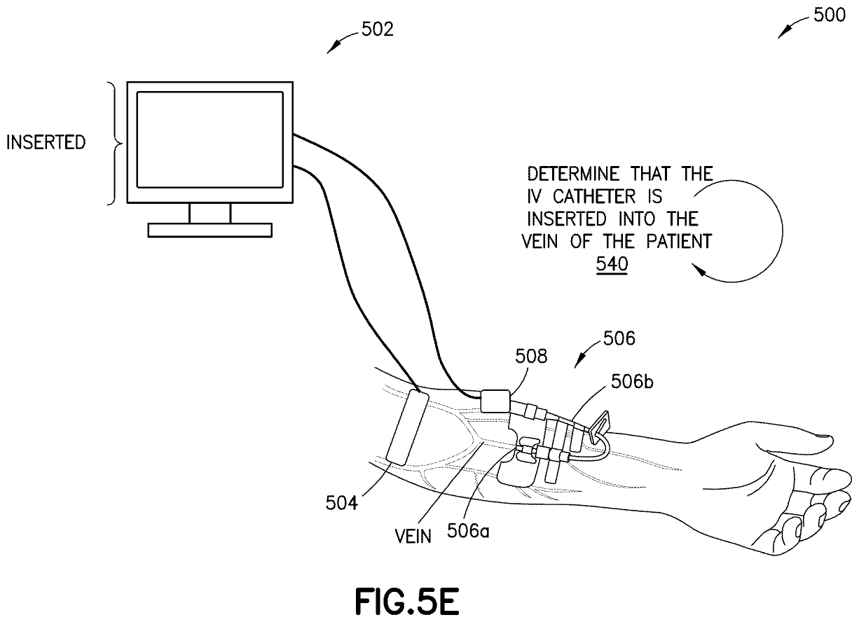

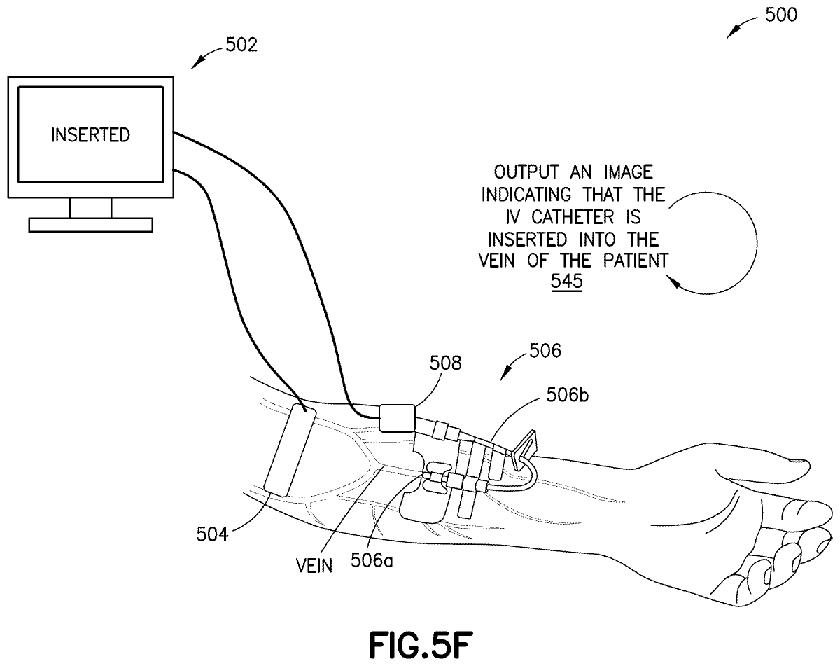

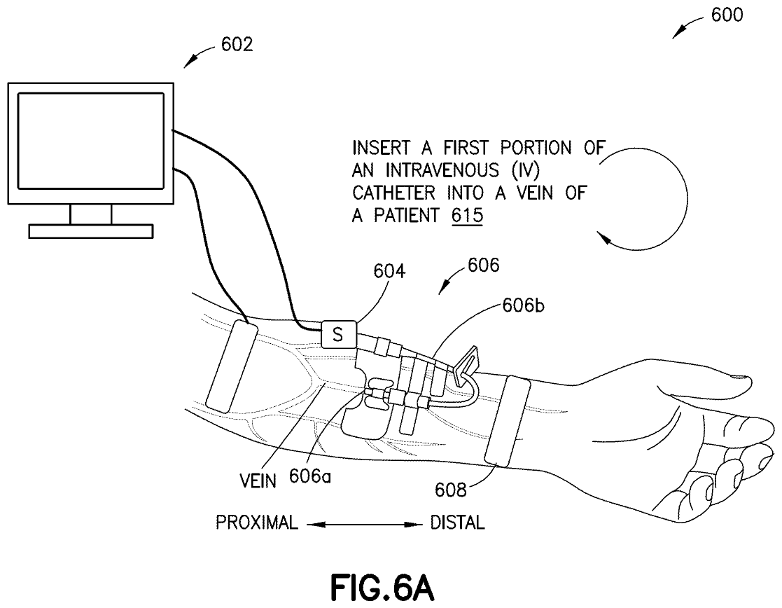

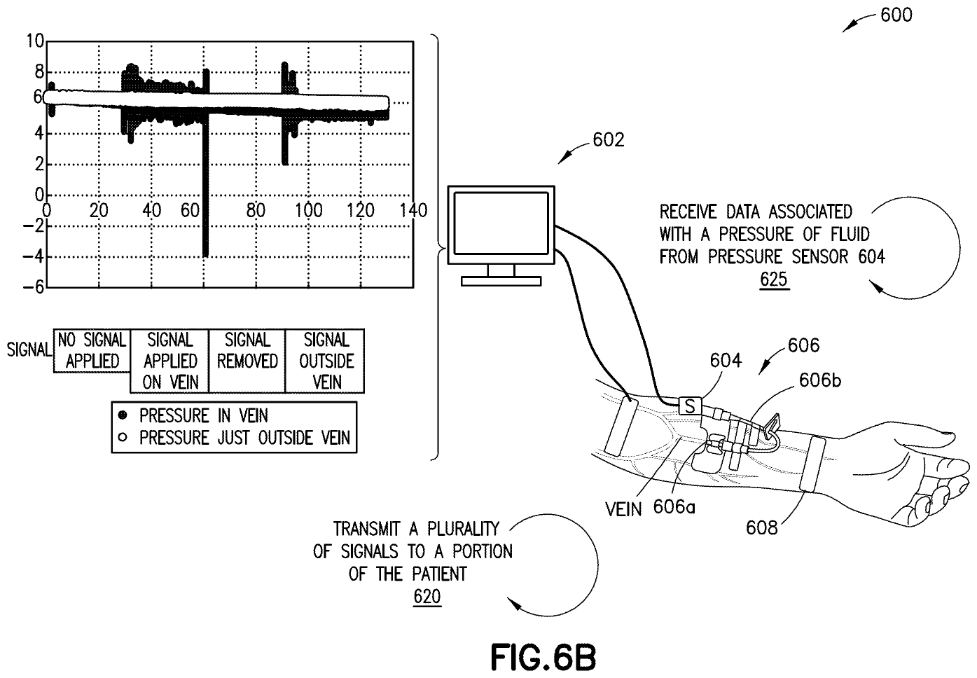

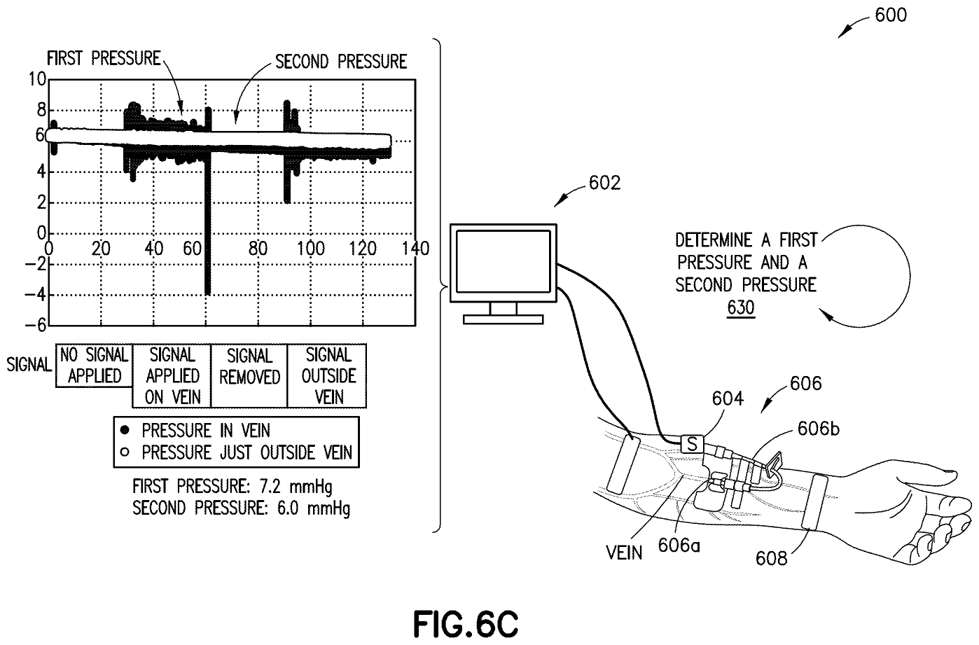

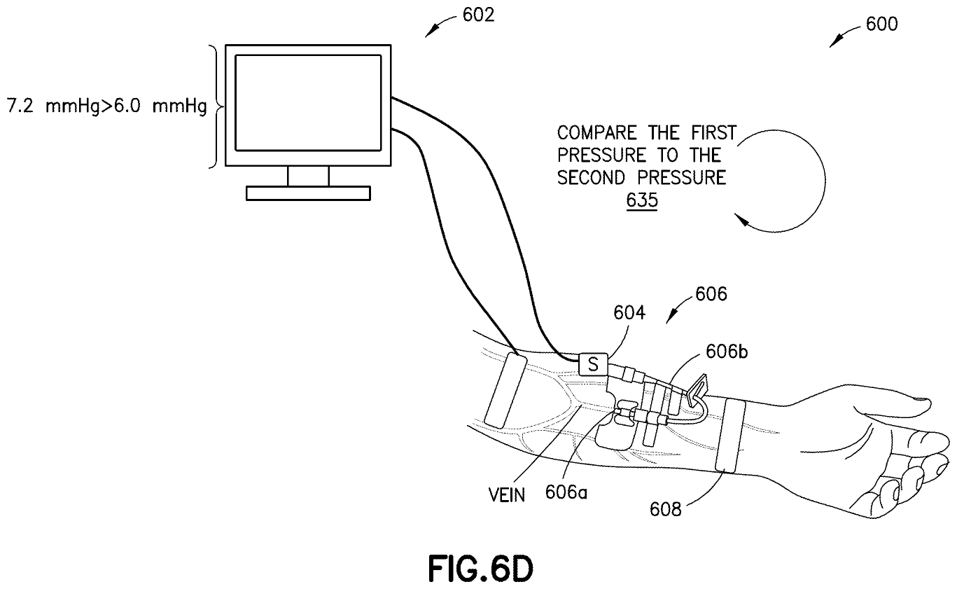

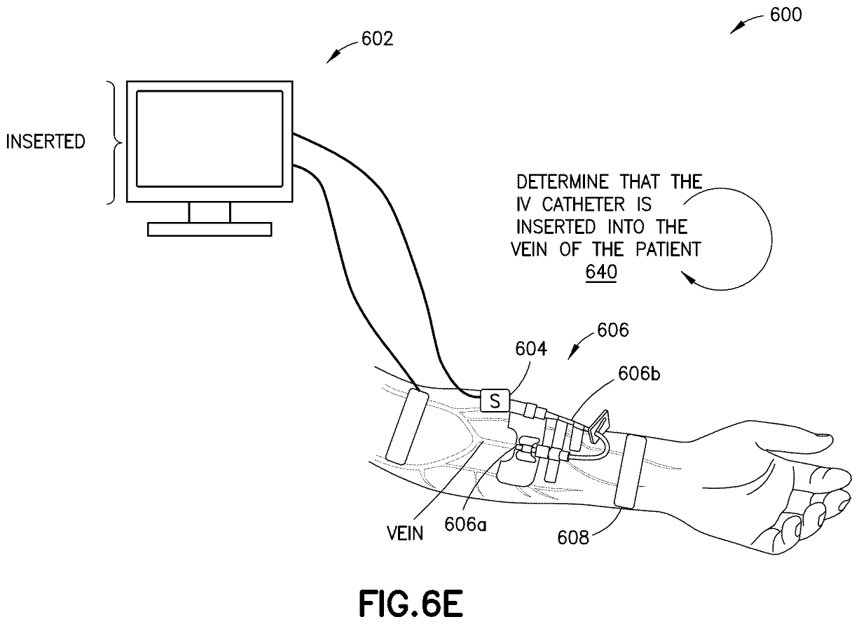

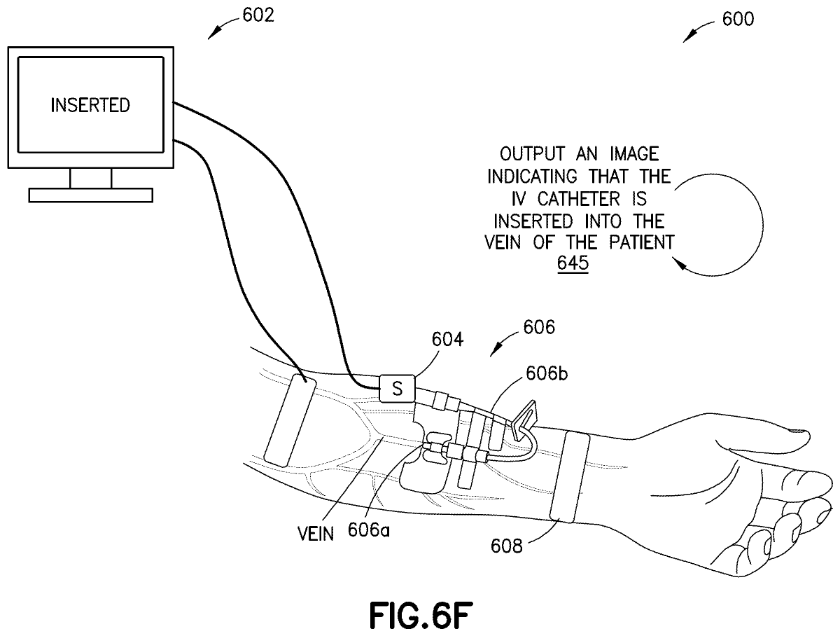

Additionally, or alternatively, where pressure sensor 104 is included in IV catheter 106, monitoring device 102 may receive data associated with the pressure of the fluid associated with IV catheter 106 based on placement of IV catheter 106 along a distal portion of the fluid path relative to placement of signal generator 108 along a proximal portion of the fluid path. (See, e.g., FIGS. 4A-4F). Additionally, or alternatively, where signal generator 108 is included in IV catheter 106, monitoring device 102 may receive data associated with the pressure of the fluid associated with IV catheter 106 based on placement of IV catheter 106 along a distal portion of the fluid path relative to placement of pressure sensor 104 along a proximal portion of the fluid path. (See, e.g., FIGS. 5A-5F). In some non-limiting embodiments or aspects, where pressure sensor 104 is included in IV catheter 106, monitoring device 102 may receive data associated with the pressure of the fluid associated with IV catheter 106 based on placement of IV catheter 106 along a proximal portion of the fluid path relative to placement of signal generator 108 along a distal portion of the fluid path. (See, e.g., FIGS. 6A-6F), In some non-limiting embodiments or aspects, where signal generator 108 is included in IV catheter 106, monitoring device 102 may receive data associated with the pressure of the fluid associated with IV catheter 106 based on placement of IV catheter 106 along a proximal portion of the fluid path relative to placement of pressure sensor 104 along a distal portion of the fluid path, (See, e.g., FIGS. 7A-7F). In some non-limiting embodiments or aspects, a proximal portion of a fluid path may be associated with an arm, a chest, a leg, a neck, a hand, a finger, a thigh, and/or the like. Additionally, or alternatively, a distal portion of a fluid path may be associated with an arm, a chest, a leg, a neck, a hand, a finger, a thigh, and/or the like.

In some non-limiting embodiments or aspects, monitoring device 102 may receive data associated with a pressure of a fluid associated with IV catheter 106 based on generation of one or more signals by signal generator 108. For example, monitoring device 102 may receive data associated with a pressure of a fluid associated with IV catheter 106 from pressure sensor 104 based on generation of one or more signals by signal generator 108 before, during, and/or after placement of IV catheter 106 (e.g., a first portion 106a of IV catheter 106) in a vein of a patient. In some non-limiting embodiments or aspects, signal generator 108 may generate the one or more signals based on signal generator 108 receiving a control signal configured to cause signal generator 108 to generate the one or more signals. For example signal generator 108 may generate the one or more signals based on signal generator 108 receiving a control signal configured to cause signal generator 108 to generate the one or more signals from monitoring device 102. In such an example, monitoring device 102 may be configured to periodically or continuously generate and transmit the control signals to signal generator 108 and monitoring device 102 may receive data associated with the pressure of the fluid associated with IV catheter 106 periodically or continuously based on monitoring device 102 transmitting the control signals to signal generator 108. Additionally, or alternatively, monitoring device 102 may be configured to generate and transmit the control signals to signal generator 108 based on receiving input from a clinician to cause monitoring device 102 to generate and transmit the control signals to signal generator 108. In some non-limiting embodiments or aspects, signal generator 108 may be configured to generate signals that may be generated by one or more anatomical features of the patient. For example, signal generator 108 may be configured to generate signals that are associated with a different frequency, different amplitude, and/or the like that are different from signals (e.g., heartbeats, repertory signals, and/or the like) generated by one or more anatomical features (e.g., the heart, lungs, and/or the like) of the patient.

As shown in FIG. 3, at step 304, process 300 may include determining whether an IV catheter is inserted into a vein of a patient. For example, monitoring device 102 may determine whether a first portion 106a of IV catheter 106 is inserted into a vein of a patient. In some non-limiting embodiments or aspects, monitoring device 102 may determine whether the first portion 106a of IV catheter 106 is inserted into a vein of a patient based on a pressure of a fluid associated with IV catheter 106. For example, monitoring device 102 may determine whether the first portion 106a of IV catheter 106 is inserted into a vein of a patient based on a pressure of a fluid associated with IV catheter 106, the pressure measured by pressure sensor 104. In such an example, the pressure of the fluid associated with IV catheter 106 may be a pressure of fluid included in at least a portion of a fluid path that a first portion 106a of IV catheter 106 is inserted in.

In some non-limiting embodiments or aspects, monitoring device 102 may determine whether IV catheter 106 is inserted into a vein of a patient based on determining that a pressure of a fluid satisfies a pressure threshold. For example, monitoring device 102 may determine whether IV catheter 106 is inserted into a vein of a patient based on determining that a pressure of a fluid measured by pressure sensor 104 satisfies a pressure threshold indicating whether a portion of an IV catheter (e.g., a first portion 106a of IV catheter 106) is inserted into a vein of a patient. In some non-limiting embodiments or aspects, the pressure threshold may be associated with a pressure (e.g., pressure measured along at least a portion of a fluid path) indicating whether the IV catheter 106 is inserted or not inserted (e.g., in a case of IV infiltration) into the vein of the patient.

In some non-limiting embodiments or aspects, monitoring device 102 may determine whether IV catheter 106 is inserted into a vein of a patient based on monitoring device 102 comparing a first pressure of a fluid and a second pressure of the fluid. For example, monitoring device 102 may receive data associated with a first pressure of a fluid measured by pressure sensor 104 and data associated with a second pressure of a fluid measured by pressure sensor 104 and monitoring device 102 may compare the first pressure of the fluid to the second pressure of the fluid. In such an example, monitoring device 102 may determine a measured deviation based on comparing the first pressure of the fluid and the second pressure of the fluid and monitoring device 102 may compare the measured deviation to a predetermined deviation threshold. In some non-limiting embodiments or aspects, the first pressure and/or the second pressure may be associated with signal generator 108 generating one or more signals to cause pressure of fluid along the fluid path to fluctuate. The predetermined deviation threshold may be associated with a deviation between a first pressure and a second pressure that indicates whether an IV catheter 106 is inserted or not inserted into the vein of the patient. In some non-limiting embodiments or aspects, monitoring device 102 may determine that IV catheter 106 is inserted into the vein of the patient based on monitoring device 102 determining that the measured deviation satisfies the pressure threshold, Additionally, or alternatively, monitoring device 102 may determine that IV catheter 106 is not inserted into the vein of the patient (e.g., that IV catheter 106 is infiltrated and/or is not in contact with the vein of the patient) based on monitoring device 102 determining that the measured deviation does not satisfy the pressure threshold.

In some non-limiting embodiments or aspects, monitoring device 102 may determine whether IV catheter 106 is inserted into the vein of the patient based on monitoring device 102 determining that the first pressure of the fluid measured by pressure sensor 104 and/or that the second pressure of the fluid measured by pressure sensor 104 is associated with (e.g., represents) a known signal, such as a frequency or a pattern. For example, monitoring device 102 may determine whether IV catheter 106 is inserted into the vein of the patient based on monitoring device 102 determining that the first pressure of the fluid measured by pressure sensor 104 and/or that the second pressure of the fluid measured by pressure sensor 104 is associated with (e.g., represents) a signal that indicates that IV catheter 106 is inserted into the vein of the patient or a signal that indicates that IV catheter 106 is not inserted into the vein of the patient. In some non-limiting embodiments or aspects, monitoring device 102 may determine whether IV catheter 106 is inserted into the vein of the patient based on monitoring device 102 determining that the first pressure of the fluid measured by pressure sensor 104 during a first time is associated with the known signal, such as a frequency or pattern, and/or a second pressure of the fluid measured by pressure sensor 104 during a second time is associated with the known signal.

Additionally, or alternatively, monitoring device 102 may determine whether IV catheter 106 is inserted into the vein of the patient based on monitoring device 102 determining that the first pressure of the fluid measured by pressure sensor 104 during a first time is not associated with the known signal and/or a second pressure of the fluid measured by pressure sensor 104 during a second time is not associated with the known signal. In some non-limiting embodiments or aspects, the first time may include a first period of time and/or the second time may include a second period of time. In some non-limiting embodiments or aspects, the known signal may be associated with signal generator 108 generating one or more signals to cause the pressure of the fluid along the fluid path to fluctuate such that the pressure of the fluid along the fluid path represents the signal. In some non-limiting embodiments or aspects, the known signal may be associated with (e.g., represent) a predetermined amount of fluctuations that, when detected by pressure sensor 104 during the first time and/or the second time, indicate whether IV catheter 106 is inserted into the vein of the patient. For example, the known signal may be associated with a predetermined amount of fluctuations that, when detected by pressure sensor 104 during the first time and/or the second time, indicate whether IV catheter 106 is inserted into the vein of the patient, where each of the fluctuations of the predetermined amount of fluctuations satisfies the pressure threshold. It is noted herein that the known signal, such as the frequency or pattern can be modified based on background noise present in an individual environment of use.

In some non-limiting embodiments or aspects, where monitoring device 102 determines that IV catheter 106 is inserted into a vein of a patient (“YES” at 304), process 300 may continue to step 302. Additionally, or alternatively, where monitoring device 102 determines that IV catheter 106 is not inserted into a vein of a patient (“NO” at 304), process 300 may continue to step 306.

As shown in FIG. 3, at step 306 (“NO” at 304), process 300 may include outputting an indication that an IV catheter is not inserted into a vein of a patient. For example, monitoring device 102 may output an indication that IV catheter 106 is not inserted into a vein of a patient. In such an example, monitoring device 102 may output the indication that IV catheter 106 is not inserted into the vein of the patient based on monitoring device 102 determining that IV catheter 106 is not inserted into the vein of the patient (“NO” at 304). In some non-limiting embodiments or aspects, monitoring device 102 may output the indication that IV catheter 106 is not inserted into the vein of the patient based on causing an output device (e.g., display 110, an audio device, and/or the like) to output the indication that IV catheter 106 is not inserted into the vein of the patient.

Additionally, or alternatively, monitoring device 102 may output an indication that IV catheter 106 is inserted into a vein of a patient. In such an example, monitoring device 102 may output the indication that IV catheter 106 is inserted into the vein of the patient based on monitoring device 102 determining that IV catheter 106 is inserted into the vein of the patient (“YES” at 304). In some non-limiting embodiments or aspects, monitoring device 102 may output the indication that IV catheter 106 is inserted into the vein of the patient based on monitoring device 102 causing an output device (e.g., display 110, an audio device, and/or the like) to output the indication that IV catheter 106 is inserted into the vein of the patient.

Referring now to FIGS. 4A-4F, FIGS. 4A-4F are perspective views of an implementation 400 of non-limiting embodiments or aspects of a system for determining whether a catheter is inserted into a vein of a patient. As illustrated in FIGS. 4A-4F, implementation 400 may include monitoring device 402, pressure sensor 404, IV catheter 406, signal generator 408, and/or display 410, In some non-limiting embodiments or aspects, monitoring device 402 may be the same as or similar to monitoring device 102, In some non-limiting embodiments or aspects, pressure sensor 404 may be the same as or similar to pressure sensor 104, In some non-limiting embodiments or aspects, IV catheter 406 may be the same as or similar to IV catheter 106, In some non-limiting embodiments or aspects, signal generator 408 may be the same as or similar to signal generator 108. In some non-limiting embodiments or aspects, display 410 may be the same as or similar to display 110.

As shown by reference number 415 in FIG. 4A, a first portion 406a of IV catheter 406 may be inserted into a vein of a patient. For example, the first portion 406a of IV catheter 406 may be inserted into the vein of the patient by a clinician such that a portion of the first portion 406a of IV catheter 406 (e.g., a tip of IV catheter 406) is positioned to be a predetermined distance (e.g., 0.01 inches, 1 inch, 2 inches, 6 inches, 1 foot, two feet, three feet, and/or the like) from signal generator 408. In such an example, signal generator 408 may be positioned (e.g., via a band) along a proximal portion of the patient's arm relative to the first portion 406a of IV catheter 406. Signal generator 408 may have an ultrasound gel applied thereon prior to positioning along the proximal portion of the patient's arm. For example, where signal generator 408 is a non-invasive device, signal generator 408 may have an ultrasound gel applied thereon prior to positioning along the proximal portion of the patient's arm. Additionally, or alternatively, signal generator 408 may have pressure applied (e.g., a band tightened) to increase force applied by signal generator 408 to the proximal portion of the patient's arm. The signal generator 408 may be configured to cause pressure in a fluid path including the vein of the patient, the first portion 406a of IV catheter 406, and a second portion 406b of IV catheter 406 to fluctuate in response to signal generator 408 generating one or more signals (e.g., mechanical pulses). The first portion 406a of IV catheter 406 may include pressure sensor 404. Pressure sensor 404 may be disposed within a first predetermined distance along the fluid path from the vein of the patient. Additionally, pressure sensor 404 may be disposed within a second predetermined distance along the fluid path from signal generator 408.

As shown by reference number 420 in FIG. 4B, signal generator 408 may transmit a plurality of signals to a portion of the patient. The plurality of signals may include a plurality of mechanical pulses. The plurality of signals may be generated continuously over one or more periods of time (e.g., pulses may be generated for a period of time of 30 seconds, then not generated for a period of time of 30 seconds), As shown by reference number 425 in FIG. 4B, monitoring device 402 may receive data associated with a pressure of fluid from pressure sensor 404. For example, monitoring device 402 may receive data associated with a pressure of fluid from pressure sensor 404 based on signal generator 408 transmitting the plurality of signals to the portion of the patient. It is noted herein that the scale of FIG. 48 is illustrative only and does not limit the scope of this invention.

As shown by reference number 430 in FIG. 4C, monitoring device 402 may determine a first pressure and a second pressure. For example, monitoring device 402 may determine a first pressure and a second pressure based on signal generator 408 transmitting a plurality of signals to the portion of the patient. In an example, monitoring device 402 may determine a first pressure based on (e.g., during) signal generator 408 transmitting of a plurality of signals to the portion of the patient and monitoring device 402 may determine a second pressure based on (e.g., before and/or after) signal generator 408 transmitting the plurality of signals to the portion of the patient. It is noted herein that the scale of FIG. 4C is illustrative only and does not limit the scope of this invention.

As shown by reference number 435 in FIG. 4D, monitoring device 402 may compare the first pressure to the second pressure. For example, monitoring device 402 may compare the first pressure (represented for illustrative purposes only as 7.2 mmHg) to the second pressure (represented for illustrative purposes only as 6 mmHg) and determine that the first pressure is greater than the second pressure. In some non-limiting embodiments or aspects, monitoring device 402 may determine that a deviation between the first pressure and the second pressure (in this instance identified as 1.2 mmHg, and corresponding to the difference between the first pressure and the second pressure) satisfies a permitted deviation threshold (e.g., 0.5 mmHg, represented for illustrative purposes only). In practice, the values for the first pressure and the second pressure are understood to encompass any suitable indication in which a difference in pressure is identified.

As shown by reference number 440 in FIG. 4E, monitoring device 402 may determine that IV catheter 406 is inserted into the vein of the patient. For example, monitoring device 402 may determine that IV catheter 406 is inserted into the vein of the patient based on monitoring device 402 comparing the first pressure to the second pressure and determining that the first pressure is greater than the second pressure (e.g., that the pressure measured by pressure sensor 404 increased based on the transmission of the plurality of signals to the portion of the patient). Additionally, or alternatively, monitoring device 402 may determine that IV catheter 406 is inserted into the vein of the patient based on monitoring device 402 determining that the deviation between the first pressure and the second pressure satisfied the permitted deviation threshold.

As shown by reference number 445 in FIG. 4F, monitoring device 402 may output an image indicating that IV catheter 406 is inserted into the vein of the patient. For example, monitoring device 402 may output an image indicating that IV catheter 406 is inserted into the vein of the patient based on monitoring device 402 determining that IV catheter 406 is inserted into the vein of the patient.

Referring now to FIGS. SA-5F, FIGS. 5A-5F are perspective views of an implementation 500 of non-limiting embodiments or aspects of a system for determining whether a catheter is inserted into a vein of a patient. As illustrated in FIGS. 5A-5F, implementation 500 may include monitoring device 502, pressure sensor 504, IV catheter 506, signal generator 508, and/or display 510. In some non-limiting embodiments or aspects, monitoring device 502 may be the same as or similar to monitoring device 102. In some non-limiting embodiments or aspects, pressure sensor 504 may be the same as or similar to pressure sensor 104. In some non-limiting embodiments or aspects, IV catheter 506 may be the same as or similar to IV catheter 106. In some non-limiting embodiments or aspects, signal generator 508 may be the same as or similar to signal generator 108, In some non-limiting embodiments or aspects, display 510 may be the same as or similar to display 110.

As shown by reference number 515 in FIG. 5A, a first portion 506a of IV catheter 506 may be inserted into a vein of a patient. For example, the first portion 506a of IV catheter 506 may be inserted into the vein of the patient by a clinician such that a portion of the first portion 506a of IV catheter 506 (e.g., a tip of IV catheter 506) is positioned to be a predetermined distance (e.g., 0.01 inches, 1 inch, 2 inches, 6 inches, 1 foot, two feet, three feet, and/or the like) from pressure sensor 504. In such an example, pressure sensor 504 may be positioned (e.g., via a band) along a proximal portion of the patient's arm relative to the first portion 506a of IV catheter 506. Pressure sensor 504 may have an ultrasound gel applied thereon prior to positioning along the patient's arm. Additionally, or alternatively, pressure sensor 504 may have pressure applied (e.g., a band tightened) to increase force applied by pressure sensor 504 to the proximal portion of the patient's arm. Signal generator 508 may be configured to cause pressure in a fluid path including the vein of the patient, the first portion 506a of IV catheter 506, and a second portion 506b of IV catheter 506 to fluctuate in response to signal generator 508 generating one or more signals (e.g., mechanical pulses), The first portion 506a of IV catheter 506 may include signal generator 508. Signal generator 508 may be disposed within a first predetermined distance along the fluid path from the vein of the patient. Additionally, signal generator 508 may be disposed within a second predetermined distance along the fluid path from pressure sensor 504.

As shown by reference number 520 in FIG. 5B, signal generator 508 may transmit a plurality of signals to a portion of the patient. The plurality of signals may include a plurality of mechanical pulses. The plurality of signals may be generated continuously over one or more periods of time (e.g. pulses may be generated for a period of time of 30 seconds, then not generated for a period of time of 30 seconds).

As shown by reference number 525 in FIG. 5B, monitoring device 502 may receive data associated with a pressure of fluid from pressure sensor 504. For example, monitoring device 502 may receive data associated with a pressure of fluid from pressure sensor 504 based on signal generator 508 transmitting the plurality of signals to the portion of the patient. It is noted herein that the scale of FIG. 5B is illustrative only and does not limit the scope of this invention.

As shown by reference number 530 in FIG. 5C, monitoring device 502 may determine a first pressure and a second pressure. For example, monitoring device 502 may determine a first pressure and a second pressure based on signal generator 508 transmitting a plurality of signals to the portion of the patient. In an example, monitoring device 502 may determine a first pressure based on (e.g., during) signal generator 508 transmitting of a plurality of signals to the portion of the patient and monitoring device 502 may determine a second pressure based on (e.g., before and/or after) signal generator 508 transmitting the plurality of signals to the portion of the patient. It is noted herein that the scale of FIG. 5C is illustrative only and does not limit the scope of this invention.

As shown by reference number 535 in FIG. 5D, monitoring device 502 may compare the first pressure to the second pressure. For example, monitoring device 502 may compare the first pressure to the second pressure and determine that the first pressure is greater than the second pressure. In some non-limiting embodiments or aspects, monitoring device 502 may determine that a deviation between the first pressure and the second pressure satisfies a permitted deviation threshold.

As shown by reference number 540 in FIG. 5E, monitoring device 502 may determine that IV catheter 506 is inserted into the vein of the patient. For example, monitoring device 502 may determine that IV catheter 506 is inserted into the vein of the patient based on monitoring device 502 comparing the first pressure to the second pressure and determining that the first pressure is greater than the second pressure (e.g., that the pressure measured by pressure sensor 504 increased based on the transmission of the plurality of signals to the second portion 506b of IV catheter 506). Additionally, or alternatively, monitoring device 502 may determine that IV catheter 506 is inserted into the vein of the patient based on monitoring device 502 determining that the deviation between the first pressure and the second pressure satisfied the permitted deviation threshold.

As shown by reference number 545 in FIG. 5F, monitoring device 502 may output an image indicating that IV catheter 506 is inserted into the vein of the patient. For example, monitoring device 502 may output an image indicating that IV catheter 506 is inserted into the vein of the patient based on monitoring device 502 determining that IV catheter 506 is inserted into the vein of the patient.

Referring now to FIGS. 6A-6F, FIGS. 6A-6F are perspective views of an implementation 600 of non-limiting embodiments or aspects of a system for determining whether a catheter is inserted into a vein of a patient. As illustrated in FIGS. 6A-6F, implementation 600 may include monitoring device 602, pressure sensor 604, IV catheter 606, signal generator 608, and/or display 610. In some non-limiting embodiments or aspects, monitoring device 602 may be the same as or similar to monitoring device 102, In some non-limiting embodiments or aspects, pressure sensor 604 may be the same as or similar to pressure sensor 104. In some non-limiting embodiments or aspects, IV catheter 606 may be the same as or similar to IV catheter 106, In some non-limiting embodiments or aspects, signal generator 808 may be the same as or similar to signal generator 108. In some non-limiting embodiments or aspects, display 610 may be the same as or similar to display 110.

As shown by reference number 615 in FIG. 6A, a first portion 606a of IV catheter 606 may be inserted into a vein of a patient. For example, the first portion 606a of IV catheter 606 may be inserted into the vein of the patient by a clinician such that a portion of the first portion 606a of IV catheter 606 (e.g., a tip of IV catheter 606) is positioned to be a predetermined distance (e.g., 0.01 inches, 1 inch, 2 inches, 6 inches, 1 foot, two feet, three feet, and/or the like) from signal generator 608. In such an example, signal generator 608 may be positioned (e.g, via a band, a finger cuff, and/or the like) along a distal portion of the patient's arm relative to the first portion 606a of IV catheter 606, Signal generator 608 may have an ultrasound gel applied thereon prior to positioning along the distal portion of the patient's arm, Additionally, or alternatively, signal generator 608 may have pressure applied (e.g., a band tightened) to increase force applied by signal generator 608 to the distal portion of the patient's arm. The signal generator 608 may be configured to cause pressure in a fluid path including the vein of the patient, the first portion 606a of IV catheter 606, and a second portion 606b of IV catheter 606 to fluctuate in response to signal generator 608 generating one or more signals (e.g., mechanical pulses). The first portion 606a of IV catheter 606 may include pressure sensor 604. Pressure sensor 604 may be disposed within a first predetermined distance along the fluid path from the vein of the patient. Additionally, pressure sensor 604 may be disposed within a second predetermined distance along the fluid path from signal generator 608.

As shown by reference number 620 in FIG. 6B, signal generator 608 may transmit a plurality of signals to a portion of the patient. The plurality of signals may include a plurality of mechanical pulses. The plurality of signals may be generated continuously over one or more periods of time (e.g. pulses may be generated for a period of time of 30 seconds, then not generated for a period of time of 30 seconds).

As shown by reference number 625 in FIG. 6B, monitoring device 602 may receive data associated with a pressure of fluid from pressure sensor 604. For example, monitoring device 602 may receive data associated with a pressure of fluid from pressure sensor 604 based on signal generator 608 transmitting the plurality of signals to the portion of the patient. It is noted herein that the scale of FIG. 6B is illustrative only and does not limit the scope of this invention.

As shown by reference number 630 in FIG. 6C, monitoring device 602 may determine a first pressure and a second pressure. For example, monitoring device 602 may determine a first pressure and a second pressure based on signal generator 608 transmitting a plurality of signals to the portion of the patient. In an example, monitoring device 602 may determine a first pressure based on (e.g., during) signal generator 608 transmitting a plurality of signals to the portion of the patient and monitoring device 602 may determine a second pressure based on (e.g., before and/or after) signal generator 608 transmitting the plurality of signals to the portion of the patient. It is noted herein that the scale of FIG. 6C is illustrative only and does not limit the scope of this invention.

As shown by reference number 636 in FIG. 6D, monitoring device 602 may compare the first pressure to the second pressure. For example, monitoring device 602 may compare the first pressure to the second pressure and determine that the first pressure is greater than the second pressure. In some non-limiting embodiments or aspects, monitoring device 602 may determine that a measured deviation between the first pressure and the second pressure satisfies a permitted deviation threshold.

As shown by reference number 640 in FIG. 6E, monitoring device 602 may determine that IV catheter 606 is inserted into the vein of the patient. For example, monitoring device 602 may determine that IV catheter 606 is inserted into the vein of the patient based on monitoring device 602 comparing the first pressure to the second pressure and determining that the first pressure is greater than the second pressure (e.g., that the pressure measured by pressure sensor 604 increased based on the transmission of the plurality of signals to the portion of the patient). Additionally, or alternatively, monitoring device 602 may determine that IV catheter 606 is inserted into the vein of the patient based on monitoring device 602 determining that the measured deviation between the first pressure and the second pressure satisfied the permitted deviation threshold.

As shown by reference number 645 in FIG. 6F, monitoring device 602 may output an image indicating that IV catheter 606 is inserted into the vein of the patient. For example, monitoring device 602 may output an image indicating that IV catheter 606 is inserted into the vein of the patient based on monitoring device 602 determining that IV catheter 606 is inserted into the vein of the patient.

Referring now to FIGS. 7A-7F, FIGS. 7A-7F are perspective views of an implementation 700 of non-limiting embodiments or aspects of a system for determining whether a catheter is inserted into a vein of a patient. As illustrated in FIGS. 7A-7F, implementation 700 may include monitoring device 702, pressure sensor 704, IV catheter 706, signal generator 708, and/or display 710. In some non-limiting embodiments or aspects, monitoring device 702 may be the same as or similar to monitoring device 102, In some non-limiting embodiments or aspects, pressure sensor 704 may be the same as or similar to pressure sensor 104. In some non-limiting embodiments or aspects, IV catheter 706 may be the same as or similar to IV catheter 106, In some non-limiting embodiments or aspects, signal generator 708 may be the same as or similar to signal generator 108. In some nonlimiting embodiments or aspects, display 710 may be the same as or similar to display 110.

As shown by reference number 715 in FIG. 7A, a first portion 706a of IV catheter 706 may be inserted into a vein of a patient. For example, the first portion 706a of IV catheter 706 may be inserted into the vein of the patient by a clinician such that a portion of the first portion 706a of IV catheter 706 (e.g., a tip of IV catheter 706) is positioned to be a predetermined distance (e.g., 0.01 inches, 1 inch, 2 inches, 6 inches, 1 foot, two feet, three feet, and/or the like) from pressure sensor 704, In such an example, pressure sensor 704 may be positioned (e.g., via a band, a finger cuff, and/or the like) along a distal portion of the patient's arm relative to the first portion 706a of IV catheter 706. Pressure sensor 704 may have an ultrasound gel applied thereon prior to positioning along the distal portion of the patient's arm, Additionally, or alternatively, pressure sensor 704 may have pressure applied (e.g., a band tightened) to increase force applied by pressure sensor 704 to the distal portion of the patient's arm. Signal generator 708 may be configured to cause pressure in a fluid path including the vein of the patient, the first portion 706a of IV catheter 706, and a second portion 706b of IV catheter 706 to fluctuate in response to signal generator 708 generating one or more signals (e.g., mechanical pulses). The first portion 706a of IV catheter 706 may include signal generator 708. Pressure sensor 704 may be disposed within a first predetermined distance along the fluid path from the vein of the patient, Additionally, pressure sensor 704 may be disposed within a second predetermined distance along the fluid path from signal generator 708.

As shown by reference number 720 in FIG. 7B, signal generator 708 may transmit a plurality of signals to a portion of the patient. The plurality of signals may include a plurality of mechanical pulses. The plurality of signals may be generated continuously over one or more periods of time (e.g., pulses may be generated for a period of time of 30 seconds, then not generated for a period of time of 30 seconds).

As shown by reference number 725 in FIG. 7B, monitoring device 702 may receive data associated with a pressure of fluid from pressure sensor 704. For example, monitoring device 702 may receive data associated with a pressure of fluid from pressure sensor 704 based on signal generator 708 transmitting the plurality of signals to the second portion 706b of IV catheter 706. It is noted herein that the scale of FIG. 7B is illustrative only and does not limit the scope of this invention.

As shown by reference number 730 in FIG. 70, monitoring device 702 may determine a first pressure and a second pressure. For example, monitoring device 702 may determine a first pressure and a second pressure based on signal generator 708 transmitting a plurality of signals to the second portion 706b of IV catheter 706, In an example, monitoring device 702 may determine a first pressure based on (e.g., during) signal generator 708 transmitting of a plurality of signals to the second portion 706b of IV catheter 706 and monitoring device 702 may determine a second pressure based on (e.g., before and/or after) signal generator 708 transmitting the plurality of signals to the second portion 706b of IV catheter 706. It is noted herein that the scale of FIG. 70 is illustrative only and does not limit the scope of this invention.

As shown by reference number 735 in FIG. 7D, monitoring device 702 may compare the first pressure to the second pressure. For example, monitoring device 702 may compare the first pressure to the second pressure and determine that the first pressure is greater than the second pressure. In some non-limiting embodiments or aspects, monitoring device 702 may determine that a measured deviation between the first pressure and the second pressure satisfies a permitted deviation threshold.

As shown by reference number 740 in FIG. 7E, monitoring device 702 may determine that IV catheter 706 is inserted into the vein of the patient. For example, monitoring device 702 may determine that IV catheter 706 is inserted into the vein of the patient based on monitoring device 702 comparing the first pressure to the second pressure and determining that the first pressure is greater than the second pressure (e.g., that the pressure measured by pressure sensor 704 increased based on the transmission of the plurality of signals to the portion of the patient). Additionally, or alternatively, monitoring device 702 may determine that IV catheter 706 is inserted into the vein of the patient based on monitoring device 702 determining that the measured deviation between the first pressure and the second pressure satisfied the permitted deviation threshold.

As shown by reference number 745 in FIG. 7F, monitoring device 702 may output an image indicating that IV catheter 706 is inserted into the vein of the patient. For example, monitoring device 702 may output an image indicating that IV catheter 706 is inserted into the vein of the patient based on monitoring device 702 determining that IV catheter 706 is inserted into the vein of the patient.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, any of these features can be combined in ways not specifically recited in the claims and/or disclosed in the specification. Although each dependent claim listed below may directly depend on only one claim, the disclosure of possible implementations includes each dependent claim in combination with every other claim in the claim set.

1. A system, comprising:
an intravenous (IV) catheter comprising a first portion configured to be inserted into a vein of a patient and a second portion configured to remain outside a patient when the first portion is inserted into the patient;
a pressure sensor configured to detect a pressure of a fluid associated with the second portion of the IV catheter; and
a signal generator configured to transmit a signal that causes a pressure of a fluid associated with the vein of the patient to fluctuate,
wherein the second portion of the IV catheter comprises the pressure sensor configured to detect the pressure of a fluid associated with the second portion of the IV catheter.

2. The system of claim 1, wherein the pressure sensor is disposed within a lumen of the second portion of the IV catheter.

3. The system of claim 1, wherein the pressure sensor is embedded in a sidewall of the second portion of the IV catheter.

4. The system of claim 1, wherein the pressure sensor is disposed along an exterior surface of the second portion of the IV catheter.

5. The system of claim 1, wherein, when the first portion of the IV catheter is inserted into the vein of the patient and the second portion of the IV catheter is configured to be in fluid communication with the first portion of the IV catheter and the vein of the patient.

6. The system of claim 1, wherein the pressure sensor is configured to detect the pressure of the fluid associated with the vein of the patient based on the pressure sensor being positioned along a proximal portion of a fluid path of the patient and a signal generator being placed along a distal portion of the fluid path of the patient.

7. The system of claim 1, wherein the pressure sensor is configured to detect the pressure of the fluid associated with the vein of the patient based on the pressure sensor being positioned along a distal portion of a fluid path of the patient and a signal generator being placed along a proximal portion of the fluid path of the patient.

8. The system of claim 1, wherein the fluid associated with the second portion of the IV catheter comprises fluid disposed within the second portion of the IV catheter, and
wherein the pressure sensor is configured to detect one or more fluctuations of the pressure of the fluid disposed within the second portion of the IV catheter based on generation of one or more signals transmitted to a second portion of the patient different from a first portion of the patient where the first portion of the IV catheter is inserted.

9. The system of claim 1, further comprising:
at least one processor programmed or configured to:
receive data associated with the pressure of the fluid associated with the second portion of the IV catheter, and
determine whether the IV catheter is inserted into the vein of the patient based on the pressure of the fluid associated with the second portion of the IV catheter.

10. The system of claim 9, wherein the at least one processor is further programmed or configured to:
transmit a control signal to cause the signal generator to generate the signal that causes pressure of fluid associated with the vein of the patient to fluctuate, and
wherein, when receiving the data associated with the pressure of the fluid associated with the second portion of the IV catheter, the at least one processor is further programmed or configured to:
receive the data associated with the pressure of the fluid associated with the second portion of the IV catheter based on transmitting the control signal to cause the signal generator to generate the signal that causes the pressure of fluid associated with the vein of the patient to fluctuate.

11. The system of claim 9, wherein, when determining whether the IV catheter is inserted into the vein of the patient based on the pressure of the fluid associated with the second portion of the IV catheter, the at least one processor is programmed or configured to:
determine that the pressure of the fluid associated with the second portion of the IV catheter satisfies a pressure threshold, and
determine whether the IV catheter is inserted into the vein of the patient based on determining that the pressure detected by the pressure sensor satisfies the pressure threshold.

12. The system of claim 9, wherein the data associated with the pressure of the fluid disposed within the second portion of the IV catheter comprises:
data associated with a first pressure of the fluid associated with the second portion of the IV catheter, and
data associated with a second pressure of the fluid associated with the second portion of the IV catheter, and
wherein, when determining whether the IV catheter is inserted into the vein of the patient based on the pressure of the fluid associated with the second portion of the IV catheter, the at least one processor is programmed or configured to:
compare the first pressure of the fluid associated with the second portion of the IV catheter to the second pressure of the fluid associated with the IV catheter, and
determine whether the first portion of the IV catheter is inserted into the vein of the patient based on comparing the first pressure of the fluid associated with the second portion IV catheter to the second pressure of the fluid associated with the second portion of the IV catheter.

13. The system of claim 12, wherein, when comparing the first pressure of the fluid associated with the second portion of the IV catheter to the second pressure of the fluid associated with the second portion of the IV catheter, the at least one processor is programmed or configured to:
determine a measured deviation between the first pressure of the fluid associated with the second portion IV catheter and the second pressure of the fluid associated with the IV catheter;
compare the measured deviation to a permitted deviation threshold associated with placement of the IV catheter in the vein of the patient; and
determine that the measured deviation satisfies the permitted deviation threshold, and
wherein, when determining whether the first portion of the IV catheter is inserted into the vein of the patient based on comparing the first pressure of the fluid associated with the second portion of the IV catheter to the second pressure of the fluid associated with the second portion of the IV catheter, the at least one processor is programmed or configured to:
determine whether the first portion of the IV catheter is inserted into the vein of the patient based on determining that the measured deviation satisfies the permitted deviation threshold.

14. The system of claim 9, further comprising:
a display configured to output an image indicating whether the IV catheter is inserted into the vein of the patient,
wherein the at least one processor is further programmed or configured to:
output, via the display, the image indicating whether the first portion of the IV catheter is inserted into the vein of the patient based on determining whether the first portion of the IV catheter is inserted into the vein of the patient.

15. A system, comprising:
an intravenous (IV) catheter comprising a first portion configured to be inserted into a vein of a patient and a second portion configured to remain outside a patient when the first portion is inserted into the patient;
a pressure sensor configured to detect a pressure of a fluid associated with the vein of the patient; and
a signal generator configured to transmit a signal that causes a pressure of a fluid associated with the vein of the patient to fluctuate,
wherein the second portion of the IV catheter comprises the signal generator configured to transmit the signal that causes a pressure of a fluid associated with the IV catheter to fluctuate.

16. The system of claim 15, wherein the signal generator is disposed within a lumen of the second portion of the IV catheter.

17. The system of claim 15, wherein the signal generator is embedded in a sidewall of the second portion of the IV catheter

18. The system of claim 15, wherein the signal generator is disposed along an exterior surface of the second portion of the IV catheter.

19. The system of claim 15, wherein, when the first portion of the IV catheter is inserted into the vein of the patient and the second portion of the IV catheter is configured to be in fluid communication with the first portion of the IV catheter and the vein of the patient.

20. The system of claim 15, wherein the signal that the signal generator is configured to transmit is configured to cause the pressure of the fluid in the vein of the patient to fluctuate when the first portion of the IV catheter is inserted into the vein of the patient.

21. The system of claim 20, wherein the signal that the signal generator is configured to transmit is configured to forego causing the pressure of the fluid in the vein of the patient to fluctuate beyond a fluctuation threshold when the first portion of the IV catheter is not inserted into the vein of the patient.

22. The system of claim 20, wherein the signal that the signal generator is configured to transmit is configured to forego causing the pressure of the fluid in the vein of the patient to fluctuate beyond a fluctuation threshold when a distal portion of the first portion of the IV catheter is not inserted into the vein of the patient.

23. The system of claim 15, wherein the pressure sensor is configured to detect the pressure of the fluid associated with the vein of the patient based on the pressure sensor being positioned along a proximal portion of a fluid path of the patient and the signal generator being placed along a distal portion of the fluid path of the patient.

24. The system of claim 15, wherein the pressure sensor is configured to detect the pressure of the fluid associated with the vein of the patient based on the pressure sensor being positioned along a distal portion of a fluid path of the patient and the signal generator being placed along a proximal portion of the fluid path of the patient.

25. The system of claim 15, wherein the pressure sensor is configured to detect the pressure of the fluid associated with the vein of the patient when the pressure sensor is disposed along a portion of the patient.

26. The system of claim 25, wherein the pressure sensor is configured to detect the pressure of the fluid associated with the vein of the patient when the pressure sensor is positioned within a first predetermined distance from the vein of the patient and when the pressure sensor is positioned within a second predetermined distance from the signal generator.

27. The system of claim 26, wherein the pressure sensor is configured to detect the pressure of the fluid associated with the vein of the patient when the pressure sensor is within a third predetermined distance to the signal generator along a fluid path between the pressure sensor and the signal generator.

28. The system of claim 15, further comprising:
at least one processor programmed or configured to:
transmit a control signal to cause the signal generator to generate the signal that causes pressure of fluid included in the vein of the patient to fluctuate,
receive data associated with the pressure of the fluid associated with the vein of the patient, and
determine whether the first portion of the IV catheter is inserted into the vein of the patient based on the pressure of the fluid associated with the vein of the patient.

29. The system of claim 28, further comprising:
wherein, when receiving the data associated with the pressure of the fluid associated with the vein of the patient, the at least one processor is further programmed or configured to:
receive the data associated with the pressure of the fluid associated with the vein of the patient based on transmitting the control signal to cause the signal generator to generate the signal that causes pressure of fluid associated with the vein of the patient to fluctuate.

30. The system of claim 28, wherein, when determining whether the IV catheter is inserted into the vein of the patient based on the pressure of the fluid associated with the vein of the patient, the at least one processor is programmed or configured to:
determine that the pressure of the fluid associated with the vein of the patient detected by a pressure sensor satisfies a pressure threshold, and
determine whether the first portion of the IV catheter is inserted into the vein of the patient based on determining that the pressure of the fluid associated with the vein of the patient detected by the pressure sensor satisfies the pressure threshold.

31. The system of claim 28, wherein the data associated with the pressure of the fluid associated with the vein of the patient comprises:
data associated with a first pressure of the fluid associated with the vein of the patient, and
data associated with a second pressure of the fluid associated with the vein of the patient, and
wherein, when determining whether the IV catheter is inserted into the vein of the patient based on the pressure of the fluid associated with the vein of the patient, the at least one processor is programmed or configured to:
compare the first pressure of the fluid associated with the vein of the patient to the second pressure of the fluid associated with the vein of the patient, and
determine whether the IV catheter is inserted into the vein of the patient based on comparing the first pressure of the fluid associated with the vein of the patient to the second pressure of the fluid associated with the vein of the patient.

32. The system of claim 31, wherein, when comparing the first pressure of the fluid associated with the vein of the patient to the second pressure of the fluid associated with the vein of the patient, the at least one processor is programmed or configured to:
determine a measured deviation between the first pressure of the fluid associated with the vein of the patient and the second pressure of the fluid associated with the vein of the patient;
compare the measured deviation to a permitted deviation threshold associated with placement of the IV catheter in the vein of the patient; and
determine that the measured deviation satisfies the permitted deviation threshold, and
wherein, when determining whether the IV catheter is inserted into the vein of the patient based on comparing the first pressure of the fluid associated with the vein of the patient to the second pressure of the fluid associated with the vein of the patient, the at least one processor is programmed or configured to:
determining whether the IV catheter is inserted into the vein of the patient based on determining that the measured deviation satisfies the permitted deviation threshold.

33. The system of claim 28, further comprising:
a display configured to output an image indicating whether the IV catheter is inserted into the vein of the patient,
wherein the at least one processor is further programmed or configured to:
output, via the display, the image indicating whether the IV catheter is inserted into the vein of the patient based on determine whether the IV catheter is inserted into the vein of the patient.

34.-47. (canceled)
